(19) United States
(12) Patent Application Publication
(10) Pub. No.: US 2026/0248576 A1
(43) Pub. Date: Aug. 27, 2026

(54) ENDOLUMINAL INSTRUMENT DRIVER

(71) Applicant: Auris Health, Inc., Santa Clara, CA (US)

(72) Inventors: Jingyi Xu, Mountain View, CA (US); Kyle Dhindsa, Santa Clara, CA (US); Nicholas J. Eyre, Sunnyvale, CA (US); Joseph L. Diamond, San Carlos, CA (US); Alex C. Spies, Redwood City, CA (US); Samuel J. Malanowski, Portland, OR (US); Bryan James Culver, Mountain View, CA (US); John Emedardo Rivera, Redwood City, CA (US)

(21) Appl. No.: 19/650,205

(22) Filed: Apr. 16, 2026

Related U.S. Application Data
(63) Continuation-in-part of application No. 19/358,393, filed on Oct. 14, 2025.
(60) Provisional application No. 63/707,697, filed on Oct. 15, 2024.
(60) Provisional application No. 63/707,182, filed on Oct. 14, 2024.
(60) Provisional application No. 63/707,190, filed on Oct. 14, 2024.

(51) Int. Cl. A61B 34/37; A61B 34/00; A61B 34/30

(52) U.S. Cl. CPC A61B 34/37; A61B 34/70; A61B 2034/301; A61B 2034/303

(57) ABSTRACT

A method includes advancing a shaft of a flexible scope via a set of feedrollers, the flexible scope having a handle mounted to a hull, pivoting the hull about a rotary joint to thereby pivot the handle of the flexible scope relative to the set of feedrollers, and translating the hull along a linear joint to thereby advance the handle toward the set of feedrollers.

### BACKGROUND

Minimally invasive medical procedures, such as laparoscopy, endoscopy, and robotically-assisted surgery, are increasingly used for the diagnosis or treatment of a variety of patient conditions. These techniques are attractive for their potential to minimize trauma to the patient, reduce recovery times, enhance surgeon precision, or facilitate new surgical approaches that may not be possible with traditional technologies. Such procedures can involve elongate instruments introduced through small incisions or natural orifices on a patient’s body to reach an anatomical site. These instruments are then manipulated to observe or interact with target anatomy within the patient using the tips of these instruments. A surgeon may control these instruments while observing a real-time camera feed of the anatomical site.

### BRIEF DESCRIPTION OF DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts an example of a surgical system.

FIG. 2 depicts an example of a physician console from the surgical system of FIG. 1.

FIG. 3 depicts an example of the surgical system of FIG. 1 configured for combined endoscopic and laparoscopic surgery.

FIG. 4 depicts an example of an anatomical site from the surgical system of FIG. 3.

FIG. 5 depicts an example of a physician console for use with the surgical system of FIG. 3, configured for displaying endoscopic and laparoscopic views.

FIG. 6 depicts an example of a surgical robot for use with the surgical system of FIG. 3, configured for manipulating rigid and flexible instruments for combined endoscopic and laparoscopic surgery.

FIG. 7 depicts an example of a robotic manipulator for use with the surgical robot of FIG. 6, configured for manipulating a rigid instrument.

FIG. 8 depicts an example of a robotic manipulator for use with the surgical robot of FIG. 6, configured for manipulating a flexible instrument.

FIG. 9A depicts an example of a distal assembly for use with the robotic manipulators of FIGS. 7-8, with a tool driver and an instrument in a coupled configuration.

FIG. 9B depicts the distal assembly of FIG. 9A, with the tool driver and the instrument in a decoupled configuration.

FIG. 10 depicts an example of a tool driver that may be used in the distal assembly of FIGS. 9A-9B.

FIG. 11 depicts an example of an endodriver that may be used with the robotic manipulator of FIG. 8.

FIG. 12 depicts an example set of flexible instruments mounted to the endodriver of FIG. 11 in a telescoping configuration.

FIG. 13A depicts an example of a loaded state of the endodriver of FIG. 11.

FIG. 13B depicts an example of a middle vertical state of the endodriver of FIG. 11.

FIG. 13C depicts an example of a middle transition state of the endodriver of FIG. 11.

FIG. 13D depicts an example of an inserted state of the endodriver of FIG. 11.

FIG. 14 depicts an example variation of the endodriver of FIG. 11.

FIG. 15A depicts an example of a retracted state of the endodriver of FIG. 13.

FIG. 15B depicts an example of an inserted state of the endodriver of FIG. 13.

FIG. 16 depicts an example variation of an endodriver having non-serial links coupled to the base of an operating table.

FIG. 17 depicts an example variation of an endodriver having non-serial links coupled to a table top.

FIG. 18 depicts an example variation of the flexible instruments of FIG. 12 in a telescoping configuration, where the endodriver is omitted and the telescoping instrument tips are visible.

FIG. 19 depicts the sheath of FIG. 18.

FIG. 20A depicts an example operation of the scope of FIG. 18 in a lower GI procedure without a sheath.

FIG. 20B depicts an example operation of the scope of FIG. 18 in a lower GI procedure with the sheath of FIGS. 18-19.

FIG. 21A depicts an example operation of the scope of FIG. 18 in an upper GI procedure without a sheath, where the scope is introduced to an open space.

FIG. 21B depicts an example operation of the scope of FIG. 18 in an upper GI procedure without a sheath, where a loop is forming.

FIG. 21C depicts an example operation of the scope of FIG. 18 in an upper GI procedure without a sheath, where retroflexion aiming is attempted.

FIG. 22A depicts an example operation of the scope of FIG. 18 in an upper GI procedure with the sheath of FIGS. 18-19, where the sheath provides support for the scope in an open space.

FIG. 22B depicts an example operation of the scope of FIG. 18 in an upper GI procedure with the sheath of FIGS. 18-19, where the sheath facilitates controlled insertion of the scope while mitigating loop formation.

FIG. 22C depicts an example operation of the scope of FIG. 18 in an upper GI procedure with the sheath of FIGS. 18-19, where the sheath increases a range of motion of the scope for retroflexion aiming.

FIG. 23 depicts the scope of FIG. 18.

FIG. 24 depicts an example set of vertebrate structures for a bending section of the scope of FIG. 23.

FIG. 25 depicts an example of a bending section of the scope in a bent state using vertebrate structures of FIG. 24.

FIG. 26A depicts a front perspective view of the working channel instrument of FIG. 18.

FIG. 26B depicts a rear perspective view of the working channel instrument of FIGS. 18 and 26A.

FIG. 27 depicts an example variation of a working channel instrument from the set of flexible instruments of FIG. 12.

FIG. 28 depicts an example of manual operation of the working channel instrument of FIG. 27.

FIG. 29A depicts an example variation of an instrument carriage for actuating the working channel instrument of FIG. 27, with a robotic actuator in an advanced state.

FIG. 29B depicts the instrument carriage of FIG. 29A, with a robotic actuator in a retracted state.

FIG. 30A depicts an example of a working channel instrument having a grasper.

FIG. 30B depicts an example of a working channel instrument having an injection needle.

FIG. 30C depicts an example of a working channel instrument having a snare.

FIG. 30D depicts an example of a working channel instrument having biopsy forceps.

FIG. 30E depicts an example of a working channel instrument having hemostatic forceps.

FIG. 31A depicts a feedroller assembly engaged with a shaft of a flexible instrument.

FIG. 31B depicts the feedroller assembly of FIG. 31A after advancing the shaft of the flexible instrument.

FIG. 31C depicts the feedroller assembly of FIG. 31B after rolling the shaft of the flexible instrument.

FIG. 32 depicts an example of an introducer.

FIG. 33 depicts an example of a drape for an endodriver.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description and appended drawings contain certain examples and configurations of this technology and are not intended to be an exhaustive disclosure of the only configurations in which the technology may be practiced. Other examples, features, aspects, embodiments, and advantages of the technology will be apparent to those skill in the art from this disclosure. As will be realized, the technology described herein is capable of other different and obvious aspects, all without departing from the inventive concepts disclosed herein. Accordingly, the drawings and descriptions should be regarded as illustrative in nature and not restrictive. In some instances, well-known structures and components are not described in detail or are shown in block diagram form to avoid obscuring concepts of this technology.

Minimally invasive procedures such as laparoscopy and endoscopy may allow a physician to control elongate instruments introduced through small incisions or natural orifices on a patient’s body to observe or interact with anatomical sites within the patient.

Laparoscopic procedures, for instance, can involve rigid surgical instruments introduced through one or more small incisions on a patient’s abdomen. These laparoscopic instruments are manipulated from outside the patient’s body through these small incisions to perform various surgical functions. The physician can perform surgical tasks, such as cutting, cauterizing, or grasping while observing a real-time camera feed of the internal anatomical site captured with one of these instruments (a rigid endoscope referred to as “laparoscope”). Because the physician views and interacts with organs or anatomical structures through small ports, rather than directly viewing such anatomical structures through a large incision, laparoscopic procedures are typically less invasive than open surgery.

Endoscopic procedures, for instance, can involve flexible instruments introduced through natural orifices on a patient, such as a mouth or perineal access point. Because these instruments are flexible, they can traverse through a lumen of a patient, which may follow a tortuous path, to reach a target anatomical site. The physician can visualize the anatomy using images obtained from a flexible endoscope to examine or diagnose conditions, or the physician may perform procedural tasks such as taking a sample or applying energy to the target site accessed with the flexible instrument. Because these techniques may involve accessing anatomy through natural orifices, they may be less invasive than laparoscopic techniques while allowing the physician to access or visualize regions of the patient anatomy that may not be readily reachable using laparoscopic techniques, such as the inside of organs like the stomach or colon.

Combining endoscopic and laparoscopic techniques can provide benefits of both approaches, with the flexibility for a physician to view and/or manipulate anatomy from endoscopic or laparoscopic perspectives. Among other things, technologies are described herein that facilitate endoscopic procedures, laparoscopic procedures, or robotic procedures. In some configurations, robotic systems and methods are described to facilitate combined use of endoscopic and laparoscopic approaches. In some configurations, a robotic system is provided that allows a single user to interact with a patient anatomy from endoscopic and laparoscopic approaches.

These and other features of the disclosed technology are further described below with respect to examples shown in the figures described below. It will be appreciated that there are various technical features and concepts disclosed herein which may be practiced independently from each other, in various combinations, or in other contexts beyond the particular examples shown and described with respect to these figures. Accordingly, these examples are explanatory in nature but should not be construed as limiting the scope of inventive subject matter to the precise examples disclosed.

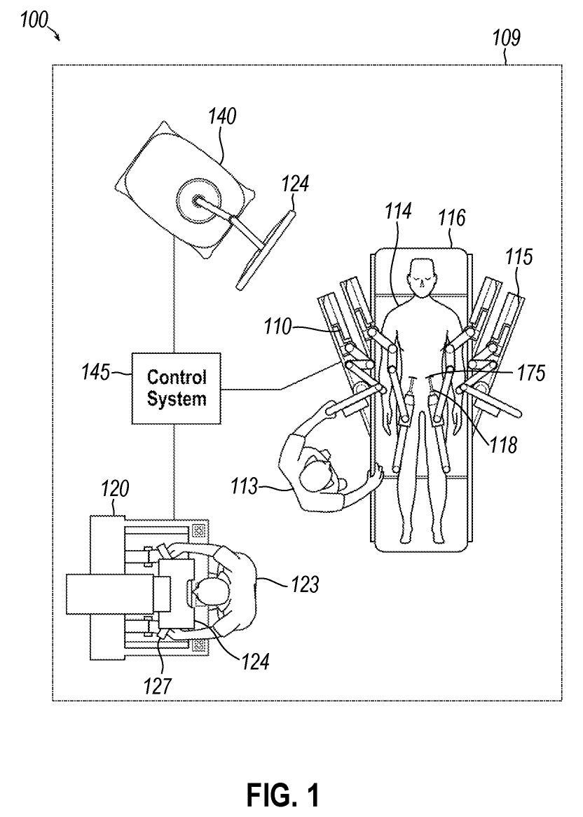

FIG. 1 depicts an example of a surgical system, in accordance with some embodiments. The surgical system 100 can be used to perform a variety of surgical procedures to diagnose and/or treat a patient 114. Examples of procedures include laparoscopy, endoscopy, thoracoscopy, urological procedures, and/or gastrointestinal (GI) procedures. As illustrated, the surgical system 100 is implemented as a robotic surgical system deployed for robotically-assisted procedures.

As seen in FIG. 1, surgical system 100 includes a surgical robot 110, a physician console 120, and a support tower 140. These components are set up in procedure area 109, such as an operating room or an endoscopy suite, and may be used in concert with each other to perform a procedure on patient 114. Components of the surgical system may be coupled physically, communicatively, and/or operatively as appropriate to facilitate operation of the surgical system 100. For instance, any two or more of the surgical robot 110, physician console 120, support tower 140, and/or other components of the surgical system may be interconnected via electrical signal lines, cabling, and/or wireless interconnections. In some configurations, components of the surgical system 100 can be situated in a common room or site. In some configurations, components of the surgical system 100 can be distributed across two or more rooms or sites that are remote from each other, where such components can be communicatively coupled over a network to implement a surgical procedure.

Surgical robot 110 is configured to interact with a patient 114 and perform various tasks. Surgical robot 110 can be actuated based on commands received from physician console 120. Such movements by the robot may be referred to as teleoperation (or telemanipulation), as they involve manipulations that are performed by the robot under human control, rather than fully autonomously. Alternatively, or in combination, surgical robot 110 can be configured to implement one or more tasks fully autonomously or semi-autonomously.

In the illustrated example, surgical robot 110 includes one or more robotic manipulators 115 configured to manipulate one or more instruments 118 (also referred to herein as “tools”). Examples of instruments include graspers, forceps, scissors, scopes, hooks, needle drivers, staplers, biopsy tools, energy delivery instruments, suction devices, irrigation devices, sheaths, snares, and various elongate instruments having flexible or rigid shafts that may be inserted into a patient’s body. In some instances, an instrument may provide a combination of two or more functions to thereby provide two or more of these instrument types in a single device. Examples of such instruments include bipolar forceps (which handle tissue and deliver energy), and suction-irrigators (which provide both suction and irrigation of fluids). Surgical robot 110 can use distal portions of instruments 118 to interact with the patient or perform various procedure tasks, such as manipulating tissue or capturing endoscopic images. In various configurations, the robotic manipulator(s) 115 may be configured to manipulate multiple different types of instruments within a particular procedure and/or across different procedures, allowing the robot to use a variety of instruments to perform a variety of surgical functions. Each of the instruments 118 may be actuatable by the surgical robot 110 in one or more degrees of freedom (DOFs) of the instrument 118 (e.g., one or more DOFs where a tip or portion of the instrument moves relative to a handle or other portion of the instrument upon actuation). In some variations, any one or more of the instruments 118 may be non-actuated.

Instruments 118 can be inserted into a body of patient 114 through one or more ports to access an anatomical site within the patient’s body. Ports may be positioned at one or more access points 175 provided by, for instance, laparoscopic incisions, cannulas, and/or natural orifices on a patient’s body to provide an access channel for the instruments 118. An instrument 118 may be introduced into the patient’s body through a port and advanced to the target anatomical site within the body. In some variations, procedures may involve one or several ports positioned at one or several access sites on the patient. In some variations, each port may be used to introduce one or multiple instruments concurrently or sequentially.

The robotic manipulators 115 can each include one or more actuators (e.g., motors) that can be electronically controlled to manipulate the instruments 118. For example, a robotic manipulator 115 can be actuated to control a position of an instrument 118 within the patient’s body and/or to actuate mechanisms of the instrument (e.g., to articulate or operate an instrument tip in one or more degrees of freedom). In some variations, surgical robot 110 includes multiple robotic manipulators 115 configured to manipulate multiple instruments 118. For example, the surgical robot 110 can include two, three, four, five, six, or more robotic manipulators 115, where each manipulator manipulates one or more corresponding instruments 118.

Each robotic manipulator 115 can include, for example, a robotic arm having a series of links connected by a series of joints. A distal end of the robotic arm can be coupled with the corresponding instrument, and a proximal end of the robotic arm can be coupled with a support of the robot. Alternatively, or in combination, a robotic manipulator can include a carriage or motorized platform that may move along a track to control an instrument or interact with patient 114. In some instances, one or more users, such as one or more members of surgical staff 113, can mount or couple various instruments 118 to the various robotic manipulators 115 during initial set up and/or throughout a procedure to exchange instruments. As instruments are mounted to the various robotic manipulators 115, surgical robot 110 can be configured to detect presence and/or identify the corresponding instruments using sensing or identification technologies, such as optical sensing, magnetic sensing, radio frequency identification (RFID), or the like.

In the example shown in FIG. 1, surgical robot 110 is configured as a table-based system, where the robotic manipulators 115 are physically coupled to or integrated with a surgical table 116 (also referred to herein as an “operating table” or “patient support”), which supports patient 114. In some variations, the surgical robot 110 can be configured as a robotic cart that can be positioned beside the patient 114 and/or beside the surgical table 116. Alternatively, or in combination, the robot can be configured as a boom-based robot, where robotic manipulators descend from an overhead boom suspended above the patient. Such an overhead boom can be supported, for example, by a cart beside the patient or from a ceiling of an operating room. In some variations, the surgical robot 110 can include one or multiple robotic carts, where each cart supports one or multiple robotic manipulators or robotic arms, and where the multiple robotic carts are configured to operate in concert with each other. For example, in some variations, a distributed or modular surgical robot can involve a modular cart system, where multiple carts are positioned beside the surgical table 116 and each cart supports a robotic manipulator 115 that manipulates a corresponding instrument 118.

Physician console 120 can be configured to provide inputs or receive outputs to or from the robot 110 or the instruments 118. As illustrated, physician console 120 includes one or more input devices 127, which a user (e.g., physician 123) can operate to provide commands for teleoperation of the robot 110. Input device 127 can include, for example, a handheld device that the physician 123 can manipulate with one or more hands to provide input to the system. In some variations, the physician console 120 can employ one or several types of input devices to provide various modes for the physician 123 to interact with the surgical system 100. Examples of input devices include pendants, gimbal-based controllers, graspers, touch sensors, trackballs, joysticks, buttons, and/or foot pedals.

Surgical system 100 can also include one or more displays 124, which can be configured to present images for observation by physician 123. For example, physician console 120 can include display 124 can be configured to display a scope view derived from endoscopic images (e.g., a video feed) captured by an instrument 118. This can facilitate control of the surgical robot 110 by the physician 123 via the input device(s) 127, while the physician views a real-time camera feed of the anatomical site within the patient’s body. Alternatively, or in combination, the display(s) 124 can be configured to display supplemental information associated with the surgical system or procedure, such as, for example, pre-operative images, navigation information, interactive menus, and/or status information associated with the instruments, the robot, and/or the surgical system. Examples of displays that may be employed by surgical system include flat panel displays, stereoscopic displays, head-mounted displays, liquid crystal displays (LCD), organic light emitting diode (OLED) displays, touch screen displays, and/or various other types of electronic display devices.

The support tower 140 can interact with surgical robot 110, instruments 118, and/or physician console 120 to provide various supporting functionality to the system, such as vision processing, fluidics, and/or energy generation. For example, the support tower 140 can process images received from an endoscope, generate light to an endoscope to illuminate the surgical site, provide suction and/or irrigation from the surgical site, operate localization sensors, such as shape sensors and/or electromagnetic (EM) sensors, and/or generate energy provided to one or more of the instruments 118 (e.g., for electrosurgery functions such as coagulating or cutting tissue). Alternatively, or in combination, support tower 140 can provide an interface for one or more users, such as surgical staff 113, to interact with the surgical system (e.g., provide inputs to the surgical system and/or observe outputs of the surgical system). In the illustrated example, support tower 140 includes one or more tower displays 124 that can be configured to present any of the same information described herein with respect to the physician console and/or additional information.

In the illustrated example, surgical robot 110, physician console 120, and support tower 140 are illustrated as separate components that may be positioned in various locations in procedure area 109. In some variations, any two or more of these components may be integral. For example, in some configurations, the support tower 140 may be provided as an integral component of the physician console 120 or surgical robot 110.

Control system 145 can be communicatively coupled to robot 110, physician console 120, and/or support tower 140. Control system 145 includes processing circuitry and memory configured to implement functions of surgical system 100, such as controlling or actuating robot 110, controlling or operating the instruments, or processing inputs or outputs to or from physician console 120. For example, processing circuitry of the control system 145 can be configured via hardware or software programming to implement any functions described further herein in connection with operation of surgical system 100, including carrying out any of the methods described herein. Examples of processing circuitry include one or more central processing units (CPUs), graphics processing units (GPUs), field programmable gate arrays (FPGAs), application specific integrated circuits (ASICs), or other processors configured to process inputs or outputs for the surgical system 100. As used herein, the term “processor” can encompass a single processing chip or integrated circuit, or multiple processing chips or integrated circuits that may be co-located or distributed in different locations and configured to execute functions described herein. Memory can store instructions that, when executed by the processor, cause the surgical system to perform any of the methods or functions described herein. As used herein, the term “memory” can encompass any suitable non-transitory processor-readable medium embodied in one or several memory devices, such as hard drives, flash memory, solid state memory, storage discs, or tapes. Components of the control system 145 may be physically located in, or physically connected to, components of the surgical system 100, such as the robot 110, the physician console 120, and/or the support tower 140. Alternatively, or in combination, components of control system 145 may be communicatively coupled to components of surgical system 100 via various wired or wireless interconnections.

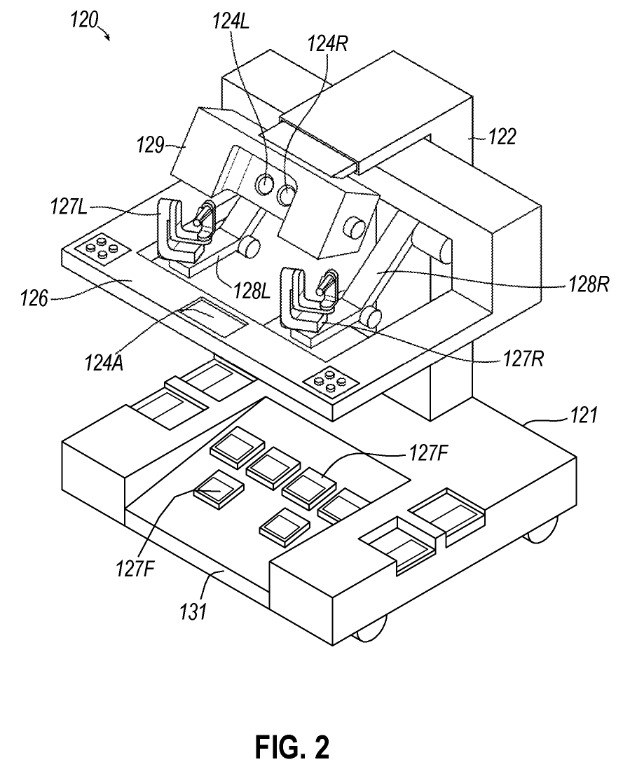

FIG. 2 depicts an example of physician console 120, in accordance with some embodiments. The physician console 120 can, for instance, be incorporated in surgical system 100 seen in FIG. 1. As noted above, physician console 120 (sometimes referred to herein as a “surgeon console”) can provide an interface for a physician (e.g., a surgeon) to interact with the surgical system 100. For example, a physician may interact with the physician console 120 to control the surgical robot and/or observe images of a surgical site.

As seen in FIG. 2, physician console 120 can include a console base 121, a pillar 122 (also referred to as a “column”) coupled to the console base 121, and a viewer assembly 129 coupled to the pillar 122. Physician console 120 further includes an armrest 126, which is coupled to and supported by pillar 122. Viewer assembly 129 can be supported by the console base 121 via the pillar and can provide a primary display for a physician to view endoscopic images of the anatomical site. In some configurations, viewer assembly 129 is configured to display anatomical images obtained from both flexible and rigid laparoscopes, as further described herein. In the illustrated example, viewer assembly 129 houses an immersive, three-dimensional, stereoscopic display, which includes a left eye display 124L and a right eye display 124R. This immersive display can present three-dimensional images to the physician when the physician inserts their head into the viewer housing. In some variations, the physician console 120 may be provided with an open design, where the viewer assembly 129 provides a two-dimensional or three-dimensional flat panel display that can present images to the physician without a need for the physician to insert their head into a viewer housing. In some variations, viewer assembly 129 may be provided by a wearable headset (e.g., a head-mounted display).

In the example shown in FIG. 2, physician console 120 also includes a pair of input devices including a left hand input device (HID) 127L and a right HID 127R, configured to be manipulated by the physician’s left and right hands, respectively (an HID is also sometimes referred to herein as a “human interface device”). Each of the HIDs can include a handle and/or finger inputs that are manipulated by a user’s hands to control a corresponding instrument and/or corresponding robotic manipulator. For example, the left HID 127L may be controlled by a user’s left hand to control a left-hand instrument manipulated by a first robotic arm of the surgical robot, and the right HID 127R may be controlled by a user’s right hand to control a right-hand instrument manipulated by a second robotic arm of the surgical robot. In the illustrated example, each of the HIDs is physically supported by an armrest 126 of the physician console 120 and/or the pillar 122 by a respective positioning arm, including a left positioning arm 128L and a right positioning arm 128R. Such positioning arms can include a series of links and series of joints, including a gimbal-based support, that supports the respective HID in space while permitting the respective HID to be manipulated in six degrees of freedom to control a corresponding position (e.g., location and/or orientation) of the respective instrument. Alternatively, or in combination, each of the left HID or right HID can include graspers and/or buttons that may be actuated by the user’s respective hands to actuate the instrument (e.g., to open or close instrument jaws) or control other functions of the surgical system. The illustrated configuration depicts grounded HIDs that are physically grounded to the console via positioning arms. In some variations, the physician console can employ ungrounded HIDs, such as free-floating and/or wireless input devices.

As seen in FIG. 2, physician console 120 can also include a foot pedal assembly 131 (e.g., a footboard) having one or more foot pedals 127F. The foot pedal(s) 127F may be coupled to or otherwise positioned at the base 121 of the physician console and may be actuated by a user’s feet to control various functions of the system. For example, in some configurations, various foot pedals may be used to perform ancillary functions of the system, such as activating energy delivery, switching control of instruments, clutching instruments, firing a staple, or toggling a menu.

In some variations, the physician console 120 can include one or more additional or secondary displays. In the illustrated example, physician console 120 includes an auxiliary display 124A, which can be implemented as a touchscreen display positioned on the armrest 126. The auxiliary display 124A can provide an additional interface for a physician to interact with the system. For example, the auxiliary display 124A can provide an additional output interface for displaying various settings or status information associated with the surgical system 100. Alternatively, or in combination, the auxiliary display 124A can provide an input interface for controlling system settings.

In some variations, it may be beneficial for a surgical system to facilitate hybrid approaches or techniques, such as the use of both flexible and rigid instrumentation and/or access to internal anatomical sites via different types of entry points. For instance, a combined endoscopic and laparoscopic system may allow for complimentary hybrid techniques that combine benefits of both laparoscopy and endoscopy.

An example of a patient condition that may benefit from combined endoscopy and laparoscopic surgery is the diagnosis or treatment of colorectal polyps. Flexible endoscopic instrumentation may access the inside of a colon through a perineal access point (e.g., anus), then be advanced to a target polyp for further examination or treatment. If the polyp is benign or can be treated without a need for surgical resection, then the procedure may proceed using a purely endoscopic technique. In cases where further intervention is needed, the procedure may then escalate to laparoscopic intervention, where rigid instruments can perform surgical manipulation and/or resection of the malignant tissue. Alternatively, or in combination, such procedures may involve concomitant use of endoscopic and laparoscopic instrumentation to target the polyp or target site, for instance, to provide laparoscopic assistance to endoscopic examination or treatment of the polyp, and/or to provide endoscopic assistance to laparoscopic examination or treatment. Such hybrid techniques may benefit from the concomitant use of instruments for viewing or manipulating the same target anatomy from different perspectives, thereby facilitating enhanced surgical techniques or improved visualization of the target site.

Various types of patient conditions and procedures may benefit from hybrid techniques or combined endoscopic and laparoscopic surgery, providing potential for improved outcomes such as reduced complications, broadened use of organ preserving technique, reduced risk of injury, and reduced length of hospital stays. Examples of procedures that may benefit from combined endoscopic and laparoscopic surgery include upper GI procedures, lower GI procedures, and thoracic procedures.

Upper GI procedures can, for instance, involve one or more flexible instruments (such as an endoscope and/or working channel tools) introduced to an anatomical site (such as the stomach or upper GI tract) through a patient’s mouth. With a combined endoscopic and laparoscopic system, one or more rigid instruments, such as a laparoscope and/or one or more other laparoscopic instruments, may access the anatomical site through one or more incisions on the patient’s abdomen to access the same anatomical site from a different perspective (for example, from outside the stomach or upper GI tract).

Lower GI procedures can, for instance, involve one or more flexible instruments introduced to an anatomical site (such as the colon or lower GI tract) through a perineal access point. With a combined endoscopic and laparoscopic system, one or more rigid instruments may access the anatomical site through one or more incisions on the patient’s abdomen to access the same anatomical site from a different perspective (for example, from outside the colon or lower GI tract).

Thoracic procedures can, for instance, involve one or more flexible instruments introduced to an anatomical site (such as the lung or pulmonary region) through a patient’s mouth. With a combined endoscopic and laparoscopic system, one or more rigid instruments may access the anatomical site through one or more incisions on the patient’s chest to access the same anatomical site from a different perspective (for example, from outside the lung or pulmonary region).

Robotic systems may facilitate these and other hybrid approaches, providing an integrated robotic platform that allows one or more users to control and/or visualize anatomy using endoscopic and laparoscopic approaches. In some variations, a single user may be able to control both endoscopic and instrumentation using a robotic surgical system.

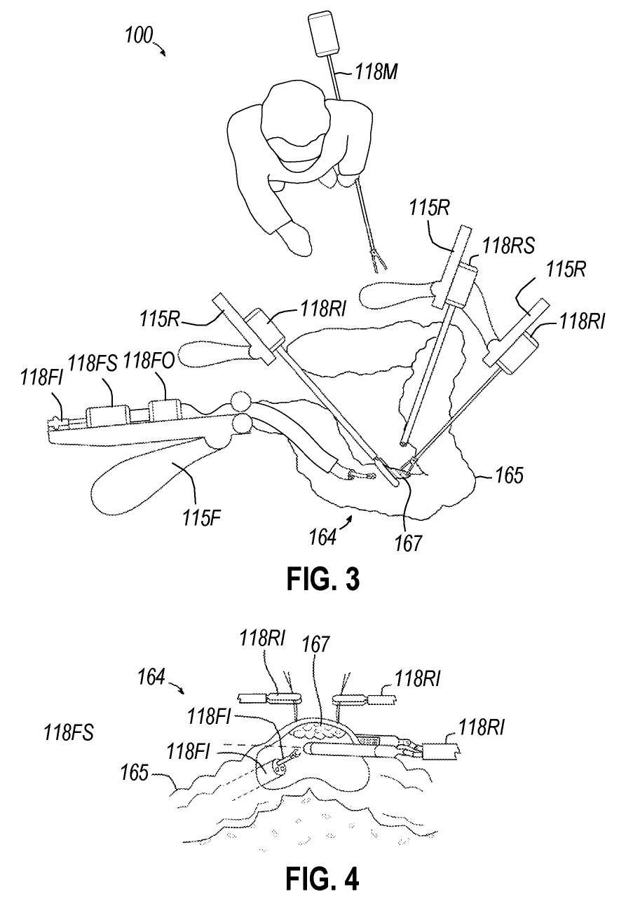

FIG. 3 depicts an example configuration of surgical system 100 in use for combined endoscopic and laparoscopic surgery, in accordance with some embodiments.

As seen in FIG. 3, surgical system 100 employs multiple robotic manipulators 115 and multiple instruments 118 interacting with an anatomical site 164. As seen in FIG. 3, anatomical site 164 encompasses an organ 165 (a colon in this example), where instruments operate on a target structure 167 within the organ (a polyp in this example) using both endoscopic (e.g., endoluminal) intervention and laparoscopic surgery.

In the illustrated example, three robotic manipulators 115R (also referred to herein as “rigid instrument manipulators” or “laparoscopic manipulators”) control three corresponding rigid instruments 118R from a laparoscopic approach, including a rigid scope 118RS (e.g., a laparoscope) for capturing images of the anatomical site, and a pair of surgical instruments 118RI (e.g., rigid laparoscopic instruments) for manipulating tissue. Rigid scope 118RS can include a camera for capturing images of the anatomical site 164 while the surgical instruments 118RI perform various tasks or manipulations on the anatomical site 164. For instance, rigid scope 118RS may include one or more image sensors arranged at its distal tip, along with corresponding optics, and a light source or light pipe at the distal tip for illuminating the anatomical site 164 while the surgical instruments 118RI operate within the field of view (FOV) of the rigid scope 118RS. In some variations, rigid scope 118RS includes a stereoscopic camera to provide three-dimensional (3D) images. In some variations, rigid scope 118RS includes a monoscopic (or “monocular”) camera to provide two-dimensional (2D) images. Each surgical instrument 118RI can include an end effector adapted for one or more tasks, such as grasping, sealing, and/or cutting, and the end effector may optionally be articulatable or actuatable by the corresponding rigid instrument manipulator 115R.

In the illustrated example, a robotic manipulator 115F (also referred to herein as a “flexible instrument manipulator” or “endoscopic manipulator”) controls three flexible instruments 118F. Here, the flexible instruments 118F are mounted to the same flexible instrument manipulator 115F in a coaxial or telescoping arrangement, and these instruments interact with the anatomical site from an endoluminal approach. The flexible instruments include an overtube 118FO, a flexible scope 118FS (e.g., a colonoscope) extending into a channel of overtube 118FO, and a working channel instrument 118FI extending into a channel of the flexible scope 118FS. Flexible scope 118FS may be robotically steerable or controllable through the lumen (colon) to reach the target structure 167. Overtube 118FO (also referred to herein as a “sheath”) may also be robotically steerable or controllable to help support or guide the flexible scope 118FS as it navigates through the lumen (colon). Working channel instrument 118FI may be introduced through a working channel of the flexible scope 118FS to interact with the target structure 167. For example, working channel instrument 118FI may be manipulated to remove tissue, apply energy, or deliver therapeutics. Flexible scope 118FS can include a camera for capturing images of the anatomical site 164 while the instruments perform various tasks or manipulations on the anatomical site 164. For instance, flexible scope 118FS may include one or more image sensors arranged at its distal tip, along with corresponding optics, and a light source or light pipe at the distal tip for illuminating the anatomical site 164. In some variations, flexible scope 118FS includes a stereoscopic camera to provide three-dimensional (3D) images. In some variations, flexible scope 118FS includes a monoscopic camera to provide two-dimensional (2D) images. In some variations, the rigid scope 118RS includes a stereoscopic (3D) camera while flexible scope 118FS includes a monoscopic (2D) camera, thereby providing enhanced visualization for precise 3D control from the laparoscopic view, while reducing cost or complexity for the flexible endoluminal camera.

In some variations, one or more manual instruments 118M may be utilized in concert with the surgical system 100. For example, a manual laparoscopic instrument may be manipulated by a beside user through a laparoscopic port. Alternatively, or in combination, one or more of the illustrated instruments may be configured for manual control, such as the working channel instrument 118FI or flexible scope 118FS.

FIG. 4 depicts an enlarged view of the anatomical site 164, to further illustrate how endoluminal and laparoscopic instruments may be used in tandem to interact with the operative site. Here, the distal tips of three surgical instruments 118RI are shown manipulating the tissue (e.g., organ 165), while rigid scope 118RS from FIG. 3 is not visible in this enlarged view. The distal tips of flexible endoscope 118FS and working channel instrument 118FI are shown observing or manipulating the target structure 167, while overtube 118FO from FIG. 3 is not visible in this enlarged view. As illustrated, the endoluminal and laparoscopic instruments may interact with the anatomical site 164 in concert with each other from different approaches, such as opposing sides of the organ wall. Here, the flexible instruments 118F observe or interact with tissue from within the organ 165, while rigid instruments 118R interact with the target from outside the organ 165. Using these instruments in concert may allow various advanced techniques, such as, for example, grasping or positioning tissue with the laparoscopic instruments to facilitate visualization of the site with the endoluminal instruments.

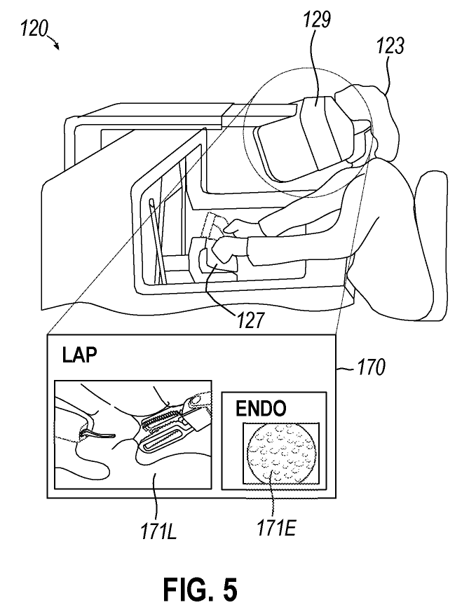

FIG. 5 depicts an example variation of physician console 120 configured for combined endoscopic and laparoscopic surgery, in accordance with some embodiments. Physician console 120 may be used, for instance, in connection with the surgical system 100 as seen in FIGS. 3-4.

As illustrated, physician console 120 may present a graphical interface 170 (also referred to herein as a “display interface”) on or more of the displays 124 (e.g., on viewer assembly 129) for viewing by physician 123. Graphical interface 170 may display one or more scope views or images of the anatomical site obtained from one or more scopes of the system. In the illustrated example, graphical interface 170 is configured to display both a laparoscopic view 171L and an endoluminal view 171E. Laparoscopic view 171L can, for instance, include one or more images obtained from rigid scope 118RS (FIG. 3). Endoluminal view 171E can, for instance, include one or more images obtained from flexible scope 118FS (FIG. 3). Physician console 120 is configured to receive input at one or more input devices 127, including commands for controlling any one or more of the instruments while the physician observes the graphical interface 170 presented on the viewer.

In the illustrated example, graphical interface 170 presents both the laparoscopic view 171L and the endoluminal view 171E concurrently (simultaneously), which may allow physician to concurrently view the anatomical site captured by the scopes from different perspectives. The concurrently displayed views may be presented, for instance, with a side-by-side configuration or a picture-in-picture configuration. In some variations, one of the views may be displayed larger than the other view or located more centrally within the display interface relative to the other view to be presented as a primary view, while the other of the views may be displayed smaller than the other view or less centrally than the other view to be presented as a secondary view. The physician console 120 may allow the user to select or switch between the views by providing input to one or more input devices, such as, for instance, one or more of the HIDs 127L, 127R, one or more of the foot pedals 127F, and/or a touch interface of the auxiliary display 124A (FIG. 2). In some variations, the views are displayed asynchronously, or not at the same time. In such variations, physician console 120 may be configured to receive input from physician 123 at any one or more of the input devices to switch between the views or select which of the views to be presented on the display.

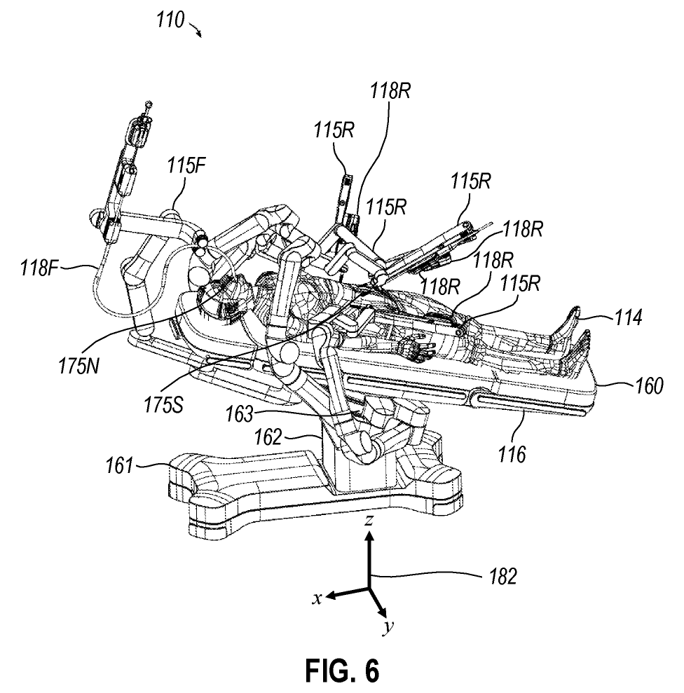

FIG. 6 depicts an example variation of surgical robot 110 configured for combined endoscopic and laparoscopic surgery, in accordance with some embodiments. Surgical robot 110 may be used, for instance, in connection with surgical system 100 as seen in FIGS. 3-4. Surgical robot 110 may be controlled, for instance, based on commands received from physician console 120 as seen in FIG. 5.

As seen in FIG. 6, surgical robot 110 includes multiple robotic manipulators 115R, 115F coupled to operating table 116 (also referred to herein as a “surgical table”). Operating table 116 includes a table base 161, a column 162 extending vertically from the table base 161, and a table top 160 supported by the column 162. Patient 114 can be supported by the operating table 116 on the table top 160, which can be movable or actuatable relative to the table base 161 and/or column 162 to adjust positioning of the patient 114 supported thereon.

The table top 160 can be adjustable to various positions, angles, or orientations relative to the table base 161 to provide a desired positioning of the patient for a given procedure or surgical task. The table top 160 can have one or more DOFs relative to table base 161, where such DOFs can be actuated by motorized mechanisms within the surgical table (e.g., one or more motors within column 162 and/or table base 161). FIG. 6 depicts a coordinate system 182 that can be used to define the DOFs of the table top 160 and/or surgical robot 110. Here, the coordinate system 182 is defined in cartesian coordinates relative to table base 161, which provides a base frame of reference that can also be aligned relative to a gravitational frame of reference of floor of procedure area 109 (FIG. 1) as the table base 161 rests on the floor. Table top 160 can have, for instance, a height DOF (also referred to as Z-height or table top lift), where the table top 160 can be raised or lowered relative to table base 161 (e.g., translation along Z-axis of coordinate system 182). Table top 160 can have a Trendelenburg DOF, where the table top 160 can be rotated about y-axis relative to table base 161 to raise or lower the foot of the table relative to the head of the table (e.g., to place the table top 160 and patient 114 in a reverse Trendelenburg position, as seen in FIG. 6, where the head of the table top 160 and patient 114 is raised relative to the foot of the table top 160 and patient 114, or to place the table top in a Trendelenburg position, where the head of the table top 160 and patient 114 is lowered relative to the foot of the table top 160 and patient 114). Table top 160 can have a tilt DOF (also referred to as “lateral tilt”), where the table top 160 can be rotated about x-axis relative to table base 161 to raise or lower the left side the table relative to the right side of the table. In some variations, table top can include more or fewer degrees of freedom than those described above. For instance, table top 160 can include any one or more DOFs associated with movement of the table top 160 relative to the table base 161. For instance, table top 160 can have any one or more of six DOFs, which include three DOFs associated with translational movement along the x, y, or z axes, respectively, and three DOFs associated with rotational movement about the x, y, or z axes, respectively.

Robotic manipulators 115 (115R, 115F) are mounted to an arm support 163, which can be coupled to the operating table 116 to provide a support base for each of the robotic manipulators 115. In the illustrated example, arm support 163 is coupled to operating table 116 distal to the Z-height, Trendelenburg, and tilt DOFs of the table top 160 such that the arm support 163, and therefore the robotic manipulators 115 (115R, 115F), move in concert with each other with unified motion as the table top 160 is actuated on those DOFs relative to the table base 161. In some variations, arm support 163 may be fixed relative to the table top 160, such that the arm support 163 and the table top 160 move in concert with each other with unified motion as the table top 160 is actuated in all of its DOFs relative to the table base 161. In some variations, arm support 163 is coupled to the column 162, the table base 161, or a cart separate from the operating table 116, in which case the arm support 163 may be movable or actuatable in any DOF independently from the table top 160. In the illustrated example, arm support 163 is positioned underneath the table top 160, such that the robotic manipulators 115 can extend around edges of the table top 160 when in a deployed configuration to position the instruments 118 in the workspace above or near the patient 114. In some variations, the robotic manipulators 115 may be movable to a stowed configuration, where the robotic manipulators are positioned beneath the table top 160 for stowage.

Robotic manipulators 115 can manipulate instruments 118 (118R, 118F) that are introduced into the patient through access points 175 (175N, 175S). In the illustrated example, surgical robot 110 includes four rigid instrument manipulators 115R that hold and manipulate four corresponding rigid instruments 118R. Each of the rigid instruments 118R may be introduced into the patient through a surgical incision 175S (e.g., a minimally invasive or laparoscopic incision). In some variations, each of the multiple rigid instruments 118R is introduced through its own respective port corresponding to its own respective incision. In some variations, two or more of the rigid instruments 118R are introduced through the same port corresponding to the same incision (e.g., for a single port surgical approach). In the illustrated example, surgical robot 110 also includes a flexible instrument manipulator 115F configured to manipulate a flexible instrument, which has a flexible shaft that may be introduced into the patient through a natural orifice 175N (e.g., a mouth). In some variations, the robotic manipulators 115 may be movable to various poses relative to the table top 160 to position the manipulators as appropriate for various procedures.

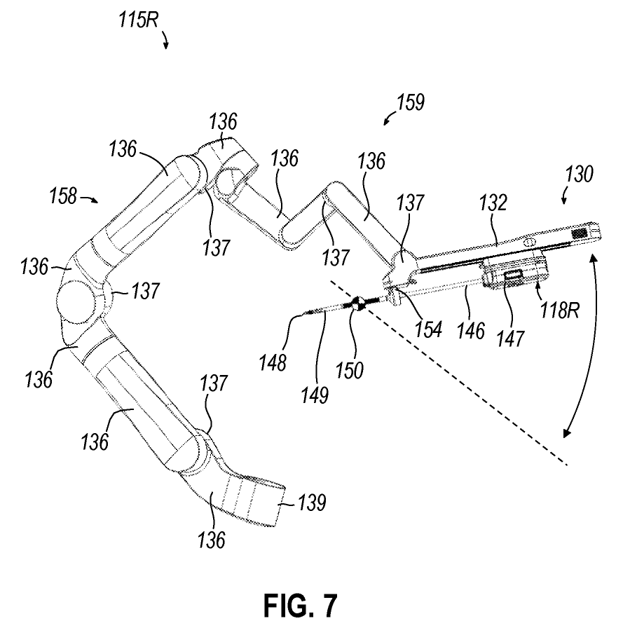

FIG. 7 depicts an example variation of rigid instrument manipulator 115R for controlling a rigid instrument 118R, in accordance with some embodiments. Rigid instrument manipulator 115R may be used, for instance, in connection with surgical robot 110 as seen in FIG. 6 and/or surgical system 100 as seen in FIG. 3.

As seen in FIG. 7, rigid instrument manipulator 115R includes a robotic arm having multiple links 136 connected by multiple joints 137. The links 136 may be arranged as a series of rigid bodies connected by joints 137 to form a kinematic chain that terminates with rigid instrument 118R. The robotic arm may include various types of joints, such as one or more pitch joints, roll joints, and/or prismatic joints, each of which may constrain movement of its adjacent links around or along certain axes relative to others. Each joint 137 may include or be coupled to an actuator (e.g., a motor), which may be actuated to control movement of adjacent links relative to one another. Each joint 137 can include or be coupled to an encoder, which can measure position information associated with the joint (e.g., a joint angle), to provide robot kinematic data. A proximal end of the robotic arm may include an arm base 139, which may be coupled to, and supported by, a mounting structure of the surgical robot, such as arm support 163 (FIG. 6). Actuation of various joints of the robotic arm can move the distal end of the robotic arm to thereby control a position of the rigid instrument 118R in space, and to move the rigid instrument 118R relative to arm base 139.

A distal assembly 130 (also referred to herein as a “distal manipulator assembly”) is arranged at the distal portion of the robotic arm. Distal assembly 130 includes a tool driver 132 (also referred to herein as an “instrument driver,” “tool holder,” or “instrument holder”) arranged at the distal end of the arm. Tool driver 132 is coupled to and supports rigid instrument 118R. Tool driver 132 is also coupled to and supports cannula 149, which is configured to receive and guide rigid instrument 118R.

The rigid instrument 118R includes an elongate shaft 146 and an instrument tip 148 arranged at a distal end of the elongate shaft 146. The elongate shaft 146 may rigidly support the instrument tip 148, which can provide an end effector for interacting with the anatomical site within the patient. Instrument shaft 146 can extend from the instrument base 147, which may provide a housing that contains mechanisms actuated by the tool driver 132 for actuating portions of the rigid instrument 118R. In some variations, the instrument tip 148 includes a robotic wrist and jaws at the distal end of the tool, which can be actuated to manipulate tissue or perform surgical tasks. In some variations, the rigid instrument 118R is non-actuated, such as some variations of a rigid laparoscope. The plurality of the joints of the robotic arm can be actuated to position and orient the tool holder 132, thereby positioning and orienting the cannula 149 and/or the instrument tip 148.

A cannula interface 154 provides a cannula holding portion of the tool driver 132. Cannula interface 154 is configured to engage the cannula 149 via, for example, a clamp, latch, or mechanical attachment, to hold and stabilize the cannula 149 with respect to the tool driver and with respect to the rigid instrument 118R mounted to the tool driver. The tool driver can include a carriage that moves along an elongate track to thereby advance or retract the instrument shaft 146 through the cannula 149. The tool driver 132 may be arranged at the distal end of a robotic arm such that articulation of the robotic arm positions and/or orients the tool driver 132 in space, thereby orienting the rigid instrument 118R and/or cannula 149.

FIG. 7 depicts an example of the rigid instrument manipulator 115R adapted for a rigid surgical instrument (e.g., a laparoscopic instrument). As seen in FIG. 7, rigid instrument manipulator 115R can manipulate or move rigid instrument 118R through a cannula 149 about a remote center of motion (RCM) 150, for example, by pivoting the rigid instrument 118R about RCM 150 in the direction of the arrow. As the elongate shaft 146 may rigidly support the instrument tip 148, moving or pivoting the instrument base 147 can cause a corresponding movement or pivoting of the instrument tip 148. The cannula 149 may, for example, provide a port that can be positioned at a small opening or incision on a patient’s body, such as incision 175S (FIG. 6), to facilitate introduction of the rigid instrument 118R to an internal anatomical site. By maintaining the position of the RCM 150 during movements of the rigid instrument 118R and/or cannula 149, the robotic manipulator can control a position of the instrument tip 148 of the rigid instrument 118R while avoiding undue trauma or stresses to the patient’s body wall as that rigid instrument 118R is moved or manipulated.

In some variations, the plurality of links 136 and joints 137 of the robotic arm can be divided into two segments. The first arm segment 158 includes a proximal set of the links 136 and joints 137, and may be referred to in some variations as a setup arm because it can position and adjust the RCM in space relative to the mounting fixture. The second arm segment 159 can include a distal set of the links 136 and joints 137, and may be referred to in some variations as the spherical arm because it can move the surgical instrument within a generally spherical workspace. The second arm segment 159 (e.g., spherical arm) can include a mechanism that mechanically constrains movement of the tool driver 132 around RCM 150, and accordingly, constrains movement of the tools mounted to the tool driver 132 (including rigid instrument 118R and cannula 149), around RCM 150. Such a mechanism may be referred to as a mechanical RCM or mechanical-based RCM. For instance, a first one of the links 136 and a second one of the links 136 of the spherical arm can be operatively coupled with a pulley mechanism to form a parallelogram that mechanically constrains movement about the RCM. The spherical arm can, for instance, have at least two degrees of freedom (DOFs). The first arm segment 158 (e.g., setup arm) can, for instance, have at least five DOFs provided by five of the joints 137 in the first segment. The proximal end of the first arm segment 158 can be mounted to an arm support (e.g., arm support 163 as seen in FIG. 6), while the distal end is coupled to the second arm segment 159.

In some variations, the robotic arm constrains motion about the RCM 150 via software or algorithms, rather than mechanical mechanisms. Such a configuration may be referred to as a software RCM or software-based RCM. In some variations, the robotic arm contains more or fewer joints and links, thereby providing more or fewer degrees of freedom for controlling motion of the robotic arm.

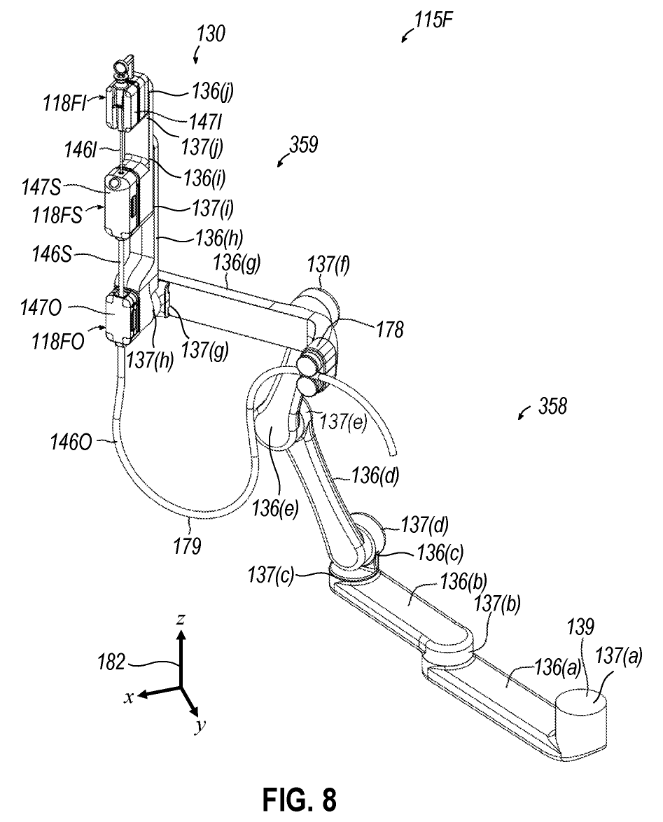

FIG. 8 depicts an example variation of flexible instrument manipulator 115F for controlling one or more flexible instruments, in accordance with some embodiments. Flexible instrument manipulator 115F may be used, for instance, in connection with surgical robot 110 as seen in FIG. 6 and/or surgical system 100 as seen in FIG. 3. Although flexible instrument manipulator 115F may share features in common with rigid instrument manipulator 115R, here the flexible instrument manipulator 115F as seen in FIG. 8 has a different architecture adapted to manipulate flexible instruments rather than rigid instruments. In some variations, flexible and rigid instruments can be manipulated by robotic manipulators having the same architecture as each other, and/or different architectures from those illustrated here.

As seen in FIG. 8, flexible instrument manipulator 115F includes a robotic arm having multiple links 136 connected by multiple joints 137. The links 136 may be arranged as a series of rigid bodies connected by joints 137 to form a kinematic chain that terminates with distal assembly 130. A proximal end of the robotic arm may include an arm base 139, which may be coupled to, and supported by, a mounting structure of the surgical robot, such as support 163 (FIG. 6). For example, actuation of various joints of the robotic arm can move the distal end of the robotic arm to thereby control a position of the flexible instrument 118F in space, and to move the flexible instrument 118F relative to arm base 139.

A distal assembly 130 is arranged at the distal portion of the robotic arm. Distal assembly 130 includes a tool driver 132 (also referred to herein as an “instrument driver”) arranged at the distal end of the arm. In this example, tool driver 132 is coupled to and supports multiple flexible instruments, which include flexible endoscope 118FS, working channel instrument 118FI, and overtube 118FO. These flexible instruments may be arranged in a coaxial or telescoping arrangement, and the tool driver 132 may include multiple instrument carriages arranged along a track to support the instrument bases of the respective instruments (e.g., working channel instrument base 147I, e.g., scope base 147S, and overtube base 147O). Each of the flexible instruments 118F includes a flexible shaft (146S, 146O, 146I) that may extend from its corresponding instrument base. Here, working channel instrument shaft 146I extends through the scope base 147S and into a working channel of the scope shaft 146S. Scope shaft 146S extends through the overtube base 147O and into a channel of the overtube shaft 146O. In some variations, the tool driver 132 may support more or fewer flexible instruments and/or different types of flexible instruments. For instance, the tool driver 132 may support one, two, three, or four flexible instruments in some variations. Each instrument base may provide a housing that contains mechanisms actuated by the tool driver 132 for actuating portions of the corresponding flexible instrument, for instance, to articulate, steer, or actuate the tip of the corresponding instrument. The plurality of the joints of the robotic arm can be actuated to position and orient the tool driver 132, thereby positioning and orienting the flexible instruments.

FIG. 8 depicts an example of the flexible instrument manipulator 115F adapted for flexible instruments (e.g., endoluminal instruments). As seen in FIG. 8, tool driver 132 can be coupled to and support feedroller assembly 178, which is configured to receive one or more of the flexible instrument shafts. Feedroller assembly 178 can include one or more feedroller wheels (e.g., a pair of opposing rollers) that can engage with the instrument shaft(s) received therein, and can be driven to advance or retract the shaft. In the illustrated example, the flexible instruments shafts are shown with a service loop portion 179, with the outermost overtube shaft 146O visible, though it will be appreciated that the scope shaft 146S and working channel tool shaft 146I can be housed within the overtube shaft 146O. The service loop portion 179 can provide slack in the instrument shaft(s). For instance, flexible instrument manipulator 115F can drive the feedroller assembly 178 in a first direction (distal or forward) to take in the service loop portion 179 and advance the shaft(s), or drive the feedroller assembly 178 in a second, opposite direction (proximal or backward) to let out the service loop portion 179 and retract the shaft(s).

In some variations, the service loop portion 179 and/or feedroller assembly 178 is omitted, in which case the flexible instrument manipulator 115F can be configured to advance or retract the instrument shaft(s) solely via motion of the manipulator itself (e.g., articulation of the arm joints and/or movement of the instrument carriages of the tool driver 132). In some variations, an additional port, such as an introducer, may be coupled to or positioned proximate to the distal assembly 130 to facilitate introducing the instrument shafts into the anatomical opening of the patient. In some variations, the robotic arm contains more or fewer joints and links, thereby providing more or fewer degrees of freedom for controlling motion of the robotic arm.

In some variations, the plurality of links 136 and joints 137 of the robotic arm can be divided into a first arm segment 358 and a second arm segment 359. The first arm segment 358 includes a proximal set of links 136(a)-(e) and proximal set of joints 137(a)-(f). The first arm segment 358 may be referred to as a deployment arm or setup arm because it can facilitate deployment of the arm from a stowed configuration, setting up the arm for a procedure, or adjusting of a position of the second arm segment 359 or feedroller assembly 178 in space relative to the mounting fixture or arm support. The second arm segment 359 can include a distal set of links 136(g)-(j) and distal set of joints 137(g)-(j), including distal assembly 130. The second arm segment may be referred to as an endodriver or endoluminal instrument driver because it can drive movement of endoluminal or flexible instruments 118F into the patient. As further described herein, the first arm segment 358 (deployment arm) can remain stationary during a procedure or during the movements and/or actuations of the flexible instruments 118F by the second arm segment (endodriver). The proximal end of the first arm segment 358 can be mounted to an arm support (e.g., arm support 163 as seen in FIG. 6) via joint 137(a), while the distal end of the first arm segment 358 can be coupled to the second arm segment 359 via joint 137(f) to support the second arm segment 359.

In some variations, the first arm segment 358 (deployment arm) can have six or more DOFs provided by six or more joints 137. In the illustrated example, six proximal joints 137(a)-(f) provide six degrees of freedom that can be actuated to deploy or stow the manipulator 115F. The arrangement of the joints 137(a)-(f) and their interconnected links 136(a)-(e) can facilitate stowage of the second arm segment (endodriver) beneath the table top 160 (FIG. 6) when not in use and deployment of the second arm segment 359 above the edge of the table top 160 to various positions for use in various procedures (e.g., at the head of the table top for an upper GI procedure or upper body approach, at the foot of the table top for a lower GI procedure or lower body approach, or laterally along a left or right side of the table top for a thoracic procedure or a lateral approach).

The arrangement of the proximal set of joints 137(a)-(f) and links 136(a)-(e) depicts an example that can facilitate deployment of the endodriver around and above the edge of a table top. As illustrated, joints 137(a)-(b) provide yaw DOFs, which can facilitate deployment of the arm horizontally or laterally (e.g., in the X-Y plane relative to coordinate system 182). The yaw DOFs can assist with reaching beyond the edge of the table top 160 (FIG. 6), where first joint 137(a) provides a DOF for adjusting yaw of a first link 136(a) relative to arm support 163 (FIG. 6), and second joint 137(b) provides a DOF for adjusting yaw of second link 136(b) relative to first link 136(a). Joint 137(c) provides a roll DOF, which can facilitate adjustments of the orientation of the endodriver relative to the patient or table top (e.g., orientation about the Z-axis of coordinate system 182), where third joint 137(c) provides a DOF for adjusting roll of third link 136(c) relative to second link 136(b). Joints 137(d)-(f) provide pitch DOFs, which can facilitate deployment of the arm vertically or upwards. The pitch DOFs arranged distal to the yaw DOFs can assist with reaching above the edge of the table top 160 (FIG. 6), where fourth joint 137(d) provides a DOF for adjusting pitch of fourth link 136(d) relative to third link 136(c), and fifth joint 137(e) provides a DOF for adjusting pitch of fifth link 136(e) relative to fourth link 136(d). A sixth joint 137(f) (e.g., the joint coupling the deployment arm to the endodriver) can provide a DOF for adjusting pitch of sixth link 136(g) relative to fifth link 136(e), which can facilitate adjustments or set up of the orientation of the endodriver relative to the patient or table top (e.g., orientation about an axis in the X-Y plane orthogonal to the Z-axis).

The second arm segment 359 (endodriver) can support the flexible instrument(s) 118F and feedroller assembly 178. The endodriver 359 can include actuators for manipulating movement of the flexible instrument(s) 118F via actuation of the joints 137 and/or actuation of drive outputs that couple to the flexible instrument(s) 118F and/or feedroller assembly 178. In some variations, the link(s) supporting the flexible instrument(s) 118F can move relative to the link(s) supporting the feedroller assembly 178 while the link(s) supporting the feedroller assembly 178 remain stationary to stabilize the feedroller assembly 178 near the orifice where the instruments are introduced into the patient. In the illustrated example, link 136(g) is coupled to and supports feedroller assembly 178, while links 136(h)-(j) are coupled to and support flexible instruments 118FO, 118FS, 118FI.

While particular arrangements of links and joints are shown and described with respect to FIG. 8, it will be appreciated that the flexible robotic manipulator 115F can take different forms and have different kinematic implementations. In some variations, the flexible robotic manipulator 115F contains more or fewer joints and links, or different types of joints or links, thereby providing more, fewer, and/or different degrees of freedom for controlling motion of the robotic arm. In some variations, the deployment arm is provided by passive lockable joints or actively controlled joints, and in some variations the flexible manipulator is 115F may not divided into first and second arm segments as described above.

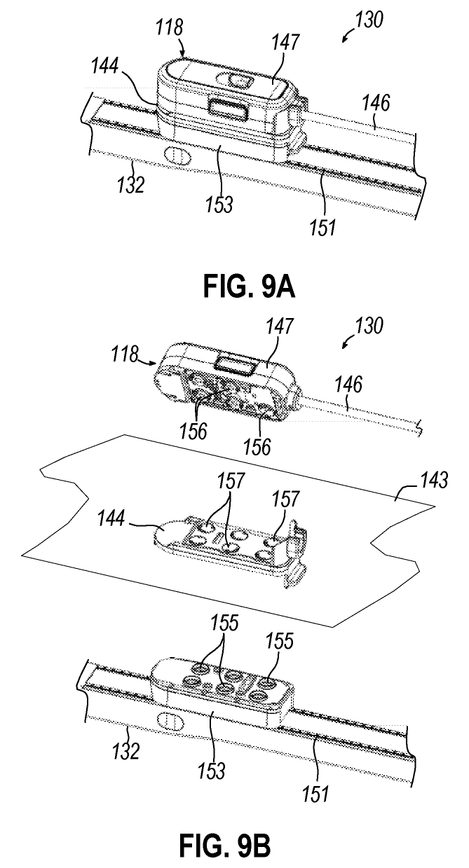

FIGS. 9A-9B depicts an example of a distal assembly 130 of a robotic manipulator, in accordance with some embodiments. FIG. 9A is an enlarged view of distal assembly 130 with instrument 118 coupled to tool driver 132. FIG. 9B depicts the distal assembly 130 in decoupled configuration, with instrument 118 detached from tool driver 132 to illustrate various interfaces therebetween. Distal assembly 130 may be used in connection with any of the robotic manipulators described herein, including, for instance, robotic manipulators 115R, 115F as seen in FIGS. 7-8. Instrument 118 may be configured in accordance with any of the instruments described herein, including, for instance, flexible and rigid instruments 118R, 118F.

As illustrated, distal assembly 130 can include a tool driver 132 coupled with instrument 118 mounted thereon. The tool driver 132 may include an elongate track 151 (also referred to herein as a “stage”) having longitudinal guides, and an instrument carriage 153, which is slidingly engaged with elongate track 151 and the longitudinal guides. The instrument carriage 153 provides an instrument holding portion configured to receive an instrument base 147 of instrument 118. An instrument base is sometimes referred to herein as a “handle” because it may be a part of the instrument configured to be held by the robot and/or a human user. Instrument carriage 153 can move along the elongate track 151 to thereby advance or retract the instrument 118.

Instrument base 147 may be coupled to the instrument carriage 153 through an adapter 144. Adapter 144 can be coupled to a drape 143, such that the adapter 144 and drape 143 provide a barrier that separates the robotic manipulator (which may be capital equipment) from the instruments (which may be consumable equipment). Such a barrier may help maintain cleanliness for the robot or sterility within the field where instruments interact with the patient. The barrier or drape 143 may include portions that extend over the robotic manipulator, robotic arm, and/or portions of the surgical robot. In some variations, instrument base 147 can be coupled to the instrument carriage 153 or tool driver 132 directly or without an adapter or barrier.

The tool driver 132 may actuate movements or functions of the instrument 118 mounted thereon. For example, tool driver 132 can actuate mechanisms in the housing of the instrument base 147 to actuate the instrument tip, such as through a cable system (e.g., pull wires) manipulated and controlled by actuated drives. The tool driver 132 may include different configurations of actuated drives. For example, as seen in FIG. 9B, instrument carriage 153 can include a set of drive outputs 155, which may engage a set of complementary drive inputs 156 on the instrument base 147 of instrument 118. The drive outputs 155 may be configured to engage the drive inputs 156 on the instrument 118 through intervening drive couplers 157 on the adapter 144, allowing the adapter to maintain a barrier while transferring torque or actuation forces from the tool driver 132 to the instrument 118. The drive outputs 155 may include, for example, rotary discs, each coupled to a corresponding actuator that can include a motor (and optionally a gear transmission and/or encoder). The drive outputs 155, drive inputs 156, and drive couplers 157 may each include various engagement features to facilitate engagement and mating between the corresponding inputs, outputs, and couplers to facilitate torque or force transfer for actuation. For example, each of the drive outputs 155, drive inputs 156, and/or drive couplers 157 may include a set of teeth, dogs, or notches that complement each other and mate with each other so that, when engaged and actuated by the tool driver 132, the engaged set of inputs, outputs, and couplers move in unison. The drive outputs 155 may be arranged in any suitable manner. For example, as seen in FIG. 9B, the tool driver 132 may include six rotary drives arranged in two rows, extending longitudinally along the instrument carriage 153. In some variations, the instrument carriage 153 includes more or fewer drive outputs, drive outputs positioned in different arrangements, and/or linear drive outputs instead of rotary discs.

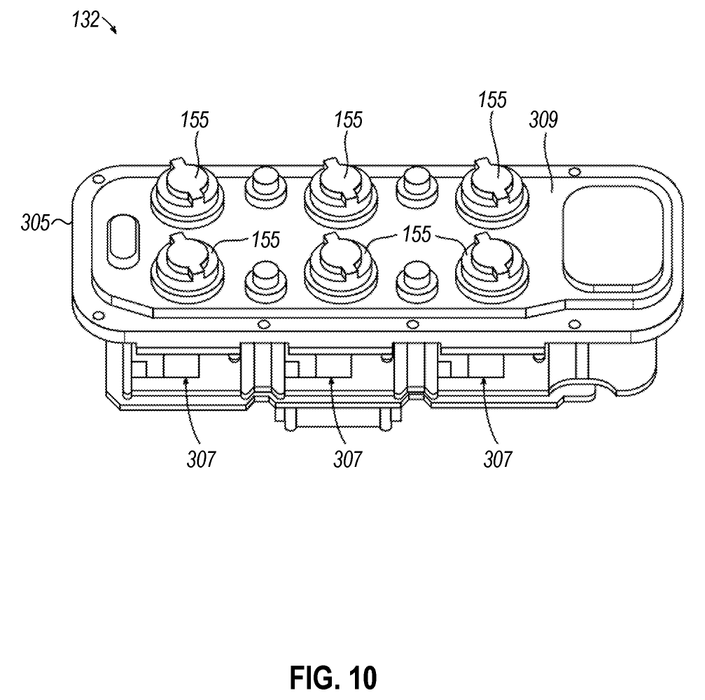

FIG. 10 depicts an example of tool driver 132 that may be incorporated into distal assembly 130, in accordance with some embodiments. Tool driver 132 is shown in FIG. 10 as an enlarged view with portions of an outer housing removed, to illustrate various internal components and details of a tool interface 309. Tool driver 132 includes a driver housing 305 and a plurality of actuators 307 housed within driver housing 305. Actuators 307 can include or be coupled to corresponding drive outputs 155 (also referred to as “output pucks”). Each actuator 307 can include a respective motor for actuating or driving rotation of the corresponding drive output 155. Drive outputs 155 are each configured to rotate relative to housing 305 to thereby drive corresponding drive inputs (e.g., drive inputs 156 of FIG. 9B). Drive outputs 155 are arranged along a face of the tool interface 309 and configured to engage with the drive inputs 156 and/or drive couplers 157 (FIG. 9B) when a tool and/or adapter is mounted to the tool interface 309. Tool driver 132 and actuators 307 can be used, for instance, to actuate flexible instruments (e.g., to steer or actuate a tool tip). Alternatively, or in combination, tool driver 132 and actuators 307 can be used to actuate feedroller assembly 178 (e.g., to drive rotation of feedroller wheels to advance or retract the shaft of the flexible instrument).

This disclosure details a kinematic implementation of an endodriver that can drive movement of a flexible or endoluminal instrument. In some embodiments, the endodriver is configured so that a portion of a scope not in the body forms a loop (service loop), and the endodriver pushes the scope into the orifice on the patient side (distal) of this loop (such as with feed rollers). When designing a robot to have a loop like this, it can be beneficial to minimize tight bends on the portion of the scope outside of the body such that the cables and structures inside are not strained impacting driving performance.

In some embodiments, the endodriver is configured to mount a scope handle and driving carriage on a member (referred to herein as a “hull”) that can pivot up and down on a rotary joint, and also translate towards and away from the feed rollers on a linear joint mounted on a member that moves minimally or not at all during the procedure (referred to herein as a “spar”). This allows the endodriver to transition between a few configurations:

When the scope is fully inserted, the hull can move parallel to the spar on the rotary joint and slide fully along the spar such that it is right up against the feed rollers. This brings the scope handle very close to the feed rollers, minimizing wasted length (the amount of scope length that cannot be inserted into the patient).

When the scope is substantially retracted, the hull can pivot up and move backwards such that it protrudes minimally beyond the end of the tabletop. In this configuration, the scope service loop bends approximately 270 degrees (one ~180 degree bend + one ~90degree bend), which is less tortuosity than if the hull remained parallel to the spar (one ~180 degree bend + two ~90 degree bends).

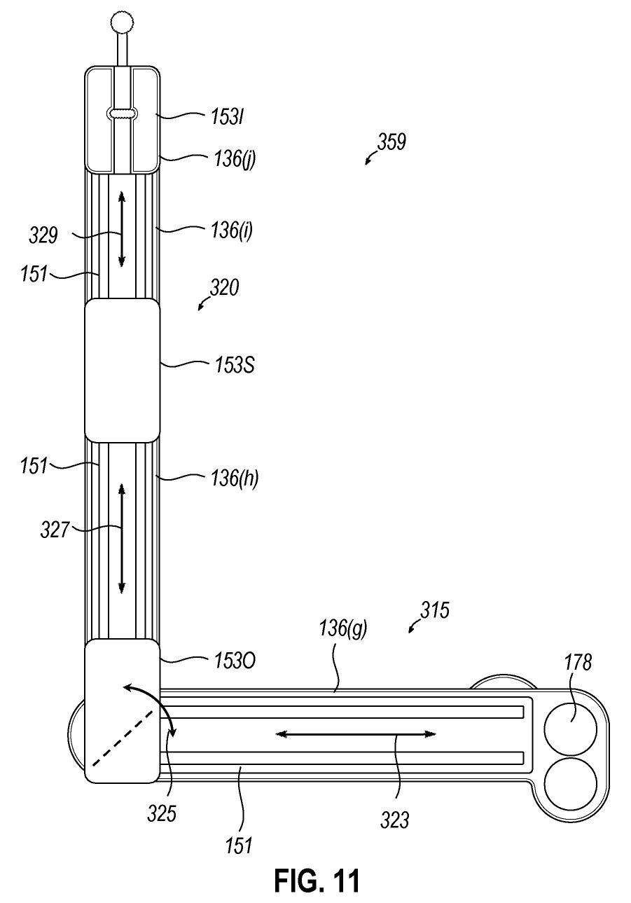

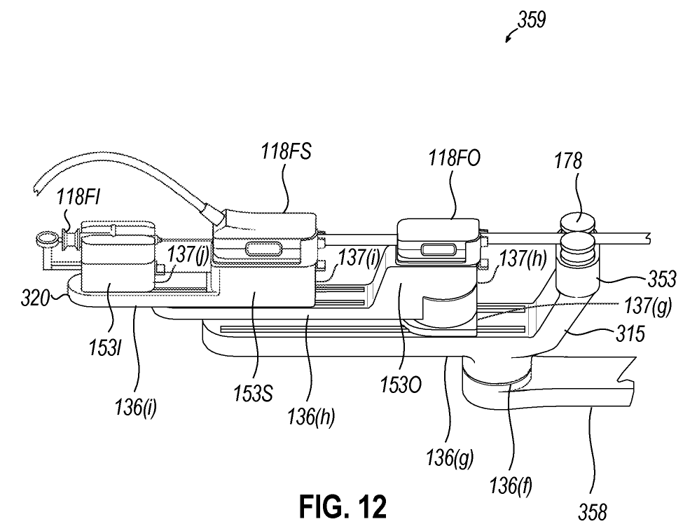

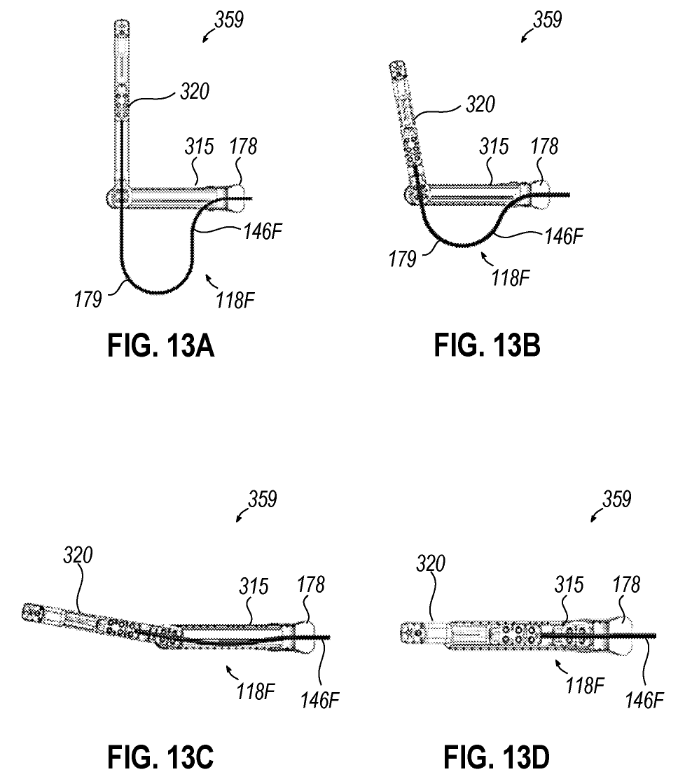

FIGS. 11-13D depict an example variation of endodriver 359, in accordance with some embodiments. FIG. 11 is a side view of the endodriver 359 depicting various degrees of freedom of the endodriver. FIG. 12 is a perspective view of the endodriver 359 depicting flexible instrumentation mounted to the endodriver 359. FIG. 13A-13D depict various states and configurations of the endodriver 359.

As seen in FIGS. 11-12, endodriver 359 can include a spar 315 and a hull 320. The spar 315 and the hull 320 are members that can each include one or more links 136, and that can be movably coupled to each other via one or more joints as further described herein. Spar 315 supports feedroller assembly 178 and can remain stationary to stabilize the feedroller assembly 178 near the orifice of the body (e.g., access point 175N of FIG. 6) as the feedroller assembly advances or retracts the flexible instrument shaft into or out of the body of the patient 114. Hull 320 supports one or more flexible instruments 118F. Hull can translate relative to spar 315 via movement along translational DOF 323 and rotate relative to spar 315 via movement about rotational DOF 325.

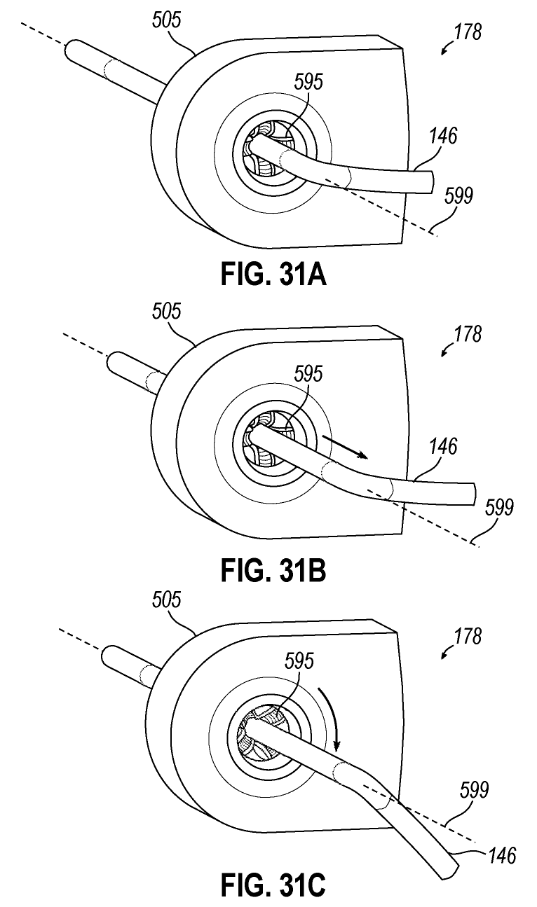

In the illustrated example, spar 315 includes link 136(g), which is coupled to and supported on deployment arm 358 via joint 136(f). Spar 315 includes feedroller coupling portion 353, which is arranged at a distal portion of link 136(h) and can house one or more actuators and corresponding drive outputs (e.g., drive outputs 155 and actuators 307 as seen in FIG. 10), and a feedroller interface for coupling to and driving feedroller assembly 178. In some variations, feedroller coupling portion 353 includes two drive outputs (and two corresponding actuators) for driving two degrees of freedom of the feedroller assembly 178 (e.g., translation and roll as described below with respect to FIGS. 31A-31C).

Hull 320 includes links 136(h)-(j), which are collectively coupled to link 136(g) via joints 137(g)-(h). As illustrated, link 136(h) (of hull 320) is rotatably coupled to link 136(g) (of spar 315) via rotation joint 137(h) and translatably coupled to link 136(g) via a linear or translation joint 137(g). The linear joint 137(g) can be controlled to drive linear movement of the link 136(h) (of hull 320) along link 136(g) (of spar 315) along translational DOF 323, such that the link 136(g) moves along track 151 along a longitudinal axis of the link 136(g). The rotation joint 137(h) can be controlled to drive rotational movement of the link 136(h) (of hull 320) about rotational DOF 325, such that the link 136(h) (of hull 320) pivots relative to the link136(h) (of spar 315) about an axis perpendicular to the linear axis of the link 136(g). Link 136(h) further supports links 136(i)-(j) such that these links move together with the link 136(h) as link 136(h) moves relative to the spar 315. In some variations, a single joint can provide for both rotation and translation of the link 136(h) (of hull 320) relative to link 136(g) (of spar 315).

In the illustrate example, hull 320 supports multiple flexible instruments via a series of links and carriages, allowing the variations instruments to be advanced or retracted relative to each other along the hull 320. Hull 320 can further support actuation of the various instruments via respective drive outputs that drive corresponding drive inputs on the instruments.

As illustrated, link 136(h) includes or is coupled to sheath carriage 153O, which provides a sheath coupling portion and interface to support and actuate sheath 118FO. Link 136(i) includes or is coupled to scope carriage 153S, which provides a scope coupling portion and interface to support and actuate sheath 153S. Link 136(i) and scope carriage 153S are translatably coupled to link 136(h) via linear joint 137(i), allowing the link 136(i) and scope carriage 153S to telescope or translate relative to link 136(h) and sheath carriage 153O along scope translation DOF 327, via movement along track 151 of link 136(h). The scope translation DOF 327 (also referred to herein as an “insertion DOF”) can be controlled to advance or retract the scope 118FS relative to sheath 118FO, for instance to extend or retract the tip of the scope 118FS relative to the tip of the sheath 118FO in a telescoping fashion.

Link 136(j) includes or is coupled to working channel instrument carriage 153I, which provides a working channel instrument coupling portion and interface to support and actuate working channel instrument 118FI. Link 136(j) and working channel instrument carriage 153I are translatably coupled to link 136(i) via linear joint 137(j), allowing the link 136(j) and working channel instrument carriage 153I to telescope or translate relative to link 136(i) and scope carriage 153S along working channel instrument translation DOF 329, via movement along track 151 of link 136(i). The working channel instrument translation DOF 329 (also referred to herein as an “insertion DOF”) can be controlled to advance or retract the working channel instrument 118FI relative to scope 118FS, for instance to extend or retract the tip of the working channel instrument 118FI relative to the tip of the scope 118FS in a telescoping fashion.

Each of the carriages may include a corresponding number of actuators and drive outputs for driving an appropriate number of DOFs for the corresponding tool or instrument. For instance, in some variations, sheath carriage 153O includes four drive outputs and corresponding actuators for actuating four DOFs of the sheath 118FO (e.g., four-way steering of the sheath tip or articulating section to provide omni-directional steering). Scope carriage 153S can include six drive outputs and corresponding actuators for actuating six DOFs of the scope 118FS (e.g., four-way steering of the scope tip, roll of the scope shaft, and an ancillary function or DOF of the scope). Working channel instrument carriage 153I can include two drive outputs and corresponding actuators for actuating two DOFs of the working channel instrument 118FS (e.g., actuation or operation of the end effector, and roll of the working channel instrument shaft). In some variations, any one or more of the carriages or instruments can include a different number of DOFs and/or corresponding outputs, for instance, to support two-way steering instead of four-way steering, to omit shaft roll, or provide other functionalities.

FIG. 11 depicts the hull 320 in a perpendicular orientation relative to spar 315, where the flexible instrument(s) 118F can be raised relative to feedroller assembly 178, thereby permitting a service loop portion 179 (e.g., FIG. 8) to hang below the spar 315 and below the feedroller assembly 178 (instruments not pictured in FIG. 11). FIG. 12 depicts the hull 320 in a parallel orientation relative to spar 315, with the instrument shafts pulled taut so that the arrangement is free of service loop portion, and the instruments are pulled closer to the feedrollers 178.

FIGS. 13A-13D depict various states of operation of the endodriver 359, to demonstrate various instrument configurations and link configurations that may be achieved by the endodriver 359.

FIG. 13A depicts an example of a loaded state of the endodriver 359, where the hull 320 (and its respective link(s)) are in a transverse orientation relative to spar 315 (and its respective link(s)) and in a retracted position relative to the feedroller assembly 178. This allows the base of flexible instrument 118F to be in an elevated position relative to feedroller assembly 178, with the flexible shaft 146F of the instrument hanging below the spar 315 and feedroller assembly 178 with service loop portion 179. The elevated and vertical configuration of the hull 320 and flexible instrument 118F can reduce an occurrence of tight bends in the instrument shaft 146F (e.g., a single 180 degree bend and 90 degree bend as seen in FIG. 13A), compared to a fully retracted position where the hull 320 remains parallel to the spar. The elevated and vertical configuration of the hull 320 and flexible instrument 118F relative to spar 315 and feedroller assembly 178 can also reduce a workspace taken up by the robotic links in an area around the patient or operating table.

FIG. 13B depicts an example of a middle vertical state of the endodriver 359, subsequent to the loaded state, where the service loop 179 has been partially taken in by feedroller assembly 178 to advance the flexible instrument shaft 146F. Here, the angle of the hull 320 relative to the spar can be made more oblique than the loaded state to reduce or alleviate bends in the service loop portion 179 of the flexible instrument shaft 146F.

FIG. 13C depicts an example of a middle transition state of the endodriver 359, subsequent to the middle vertical state, where the service loop 179 has been taken in further by feedroller assembly 178 to further advance the flexible instrument shaft 146F such that the service loop is fully or almost fully taken in. Here, the angle of the hull 320 relative to the spar can be made parallel or more oblique to substantially eliminate the bends in the portion of the flexible instrument shaft 146F between the base of the flexible instrument and the feedroller assembly 178. The base of the flexible instrument 118F can also be level or substantially level with the feedroller assembly 178 in a vertical direction (e.g., Z-axis of the coordinate system 182 of FIG. 8).

FIG. 13D depicts an example of an inserted state of endodriver 359, subsequent to the middle transition state, where the hull 320 (and its respective link(s)) are in a parallel orientation relative to spar 315 (and its respective link(s)) and in an advanced position relative to the feedroller assembly 178. This allows the base of flexible instrument 118F to be in a level position relative to feedroller assembly 178, with the flexible shaft 146F of the instrument taut and free of the service loop. The advanced and parallel configuration of the hull 320 and flexible instrument 118F relative to spar 315 and feedroller assembly 178 can reduce a wasted length of the flexible instrument (length of flexible shaft 146F outside the body) compared to a full retracted position or a configuration where the hull 320 is not translatable relative to the spar 315. The advanced position can also reduce a workspace taken up by the robotic links in an area around the patient or operating table.

In the illustrated states of FIG. 13A-13C, rotational motion of the hull 320 (e.g., about rotational DOF 325 of FIG. 11) and translational motion of the hull 320 (e.g., along translational DOF 323) can occur sequentially, such that the hull 320 is rotated to a parallel position with the spar 315 before advancing the hull 320 to an advanced position relative to spar 315. In some variations, translational and rotational motion can occur in tandem to provide combined motion.

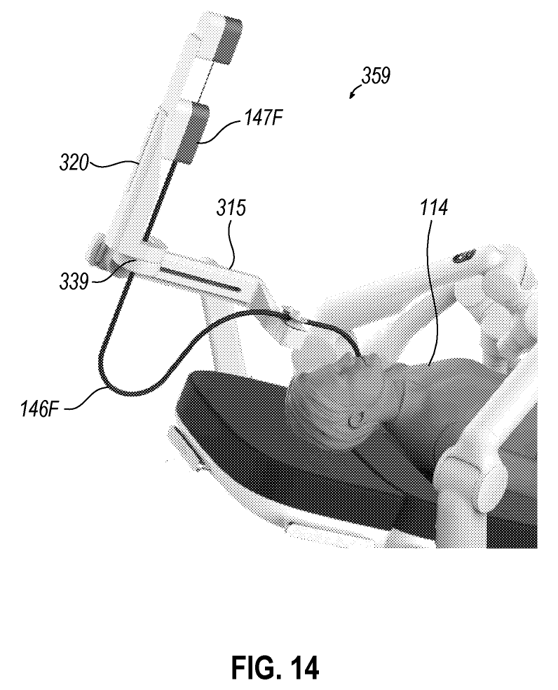

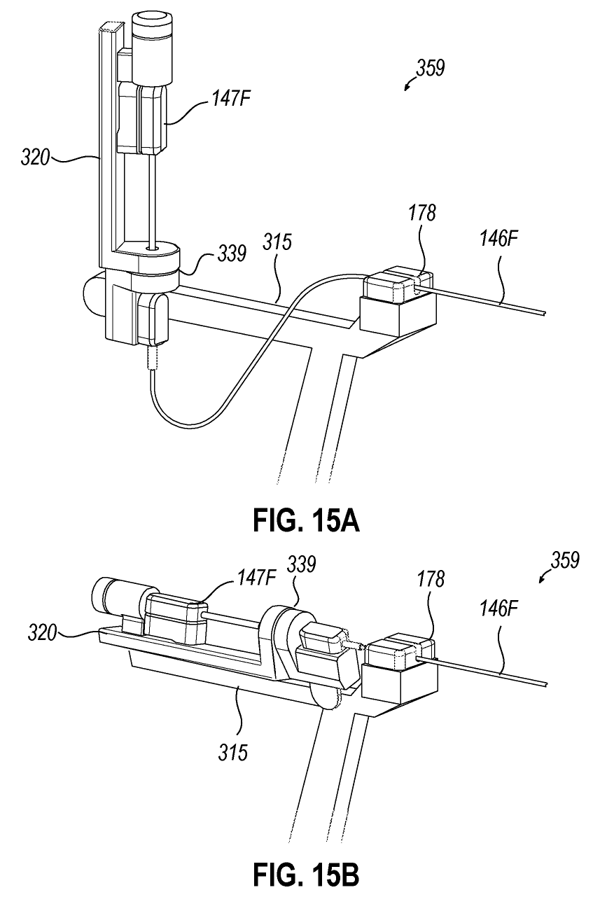

FIGS. 14-15B depict an example variations of the endodriver 359. FIG. 14 depicts endodriver 359 with the flexible instrument shaft 146F introduced into patient 114. FIGS. 15A-15B depict loaded and inserted states of the endodriver 359, respectively.

The illustrated examples may be similar to the variations described above with respect to FIGS. 11-13C, including a hull 320 that may rotate and translate relative to the spar 315 with the various degrees of freedom, links, and joints constructed similarly. In the example variations depicted in FIGS. 14-15B, the endodriver 359 includes an additional rotational joint 339 between the hull 320 and the spar 315 to provide an additional rotational degree of freedom of the hull 320 relative to the spar 315. The additional rotational joint 339 can be controlled to drive roll of the flexible instrument shaft 146F, by driving rotation of the entire link supporting instrument base 147F about the roll axis (longitudinal axis) of the instrument shaft 146F. In such configurations, a roll degree of freedom within the instrument itself may be omitted.

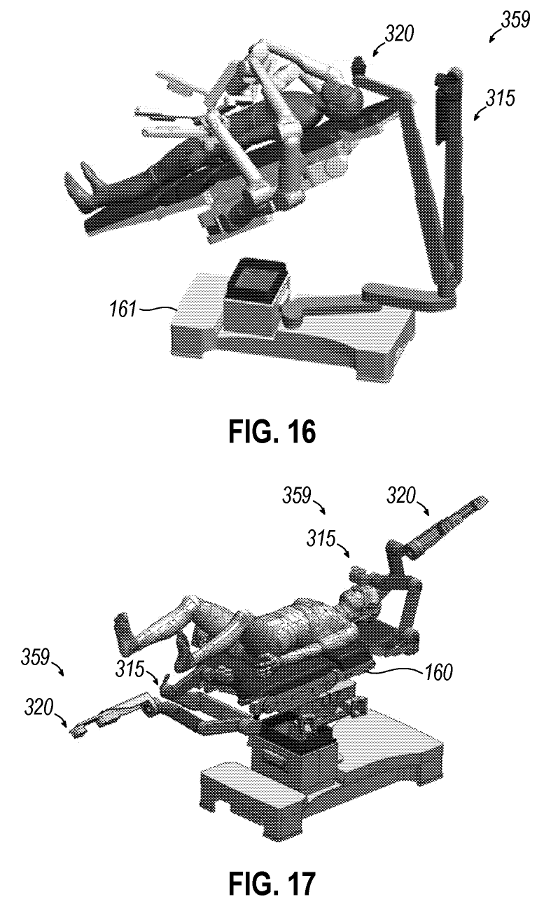

FIGS. 16-17 depict example variations of endodriver 359. The illustrated examples may be similar to the variations of the endodrive 359, except that the spar 315 (feedroller) links and the hull 320 (instrument) links are provided on separated, non-serial links of respective kinematic chains. FIG. 16 depicts an example where the endodriver 359 is coupled to the table base 161, while FIG. 17 depicts an example where the endodriver 359 is coupled to the table top 160. The example variations of endodriver 359 shown in FIGS. 16-17 may otherwise move the links and instruments in a similar fashion to the various states described above, where in a loaded state the link(s) of the non-serial hull 320 are perpendicular and retracted relative to the spar 315 and feedrollers, and where in an inserted state the link(s) of the non-serial hull 320 are in a parallel and advanced state relative to the spar 315 and feedrollers. In some variations, the separated and non-serial nature of the endodriver variations shown in FIGS. 16-17 allows for different, unconstrained movements relative to the coupled serial chain variations described above with respect to FIGS. 11-15B.

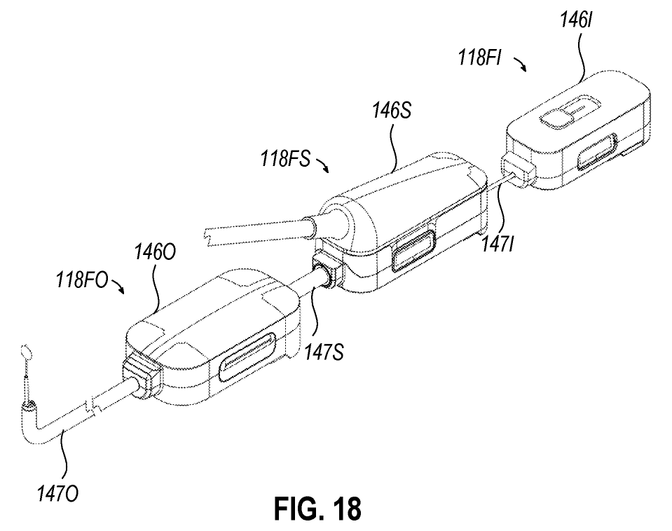

FIG. 18 depicts an example variation of the flexible instruments (118FO, 118FS, 118FI) seen in FIG. 12, with the instruments arranged in a telescoping configuration. These flexible instruments may be arranged in a coaxial or telescoping arrangement. Each of the flexible instruments 118F includes a flexible shaft (146O, 146S, 146I) that may extend from its corresponding instrument base (147O, 147S, 147I). Here, working channel instrument shaft 146I extends through the scope base 147S and into a working channel of the scope shaft 146S. Scope shaft 146S extends through the overtube base 147O and into a channel of the overtube shaft 146O. Each instrument base may provide a housing that contains mechanisms for actuating DOFs of the corresponding flexible instrument, for instance, to articulate, steer, or actuate the tip of the corresponding instrument.

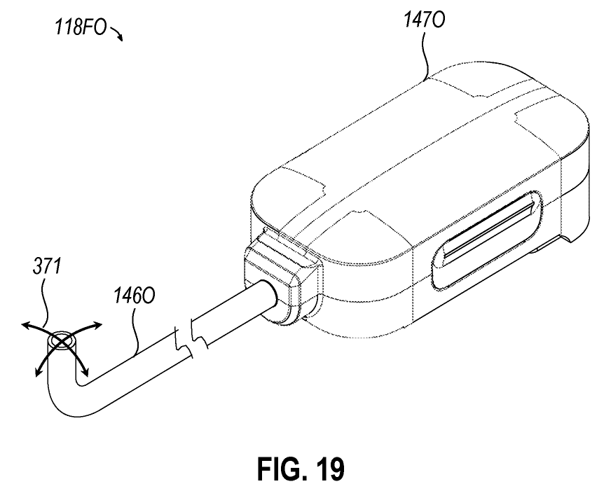

FIG. 19 depicts the sheath 118FO of FIG. 18 (also referred to as “overtube”). The sheath 118FO has an instrument base 147O and a flexible shaft 146O extending from the instrument base 147O. The instrument base 147O can house mechanisms (e.g., spools or pulleys coupled to pull wires) for steering the tip of the instrument in four steering DOFs 371, causing a distal articulating section of the instrument shaft 146O to bend relative to a proximal portion of the instrument shaft 146O and the instrument base 147O. Flexible shaft 146O of the sheath also includes a channel or lumen that can receive the shaft of the scope as noted above.

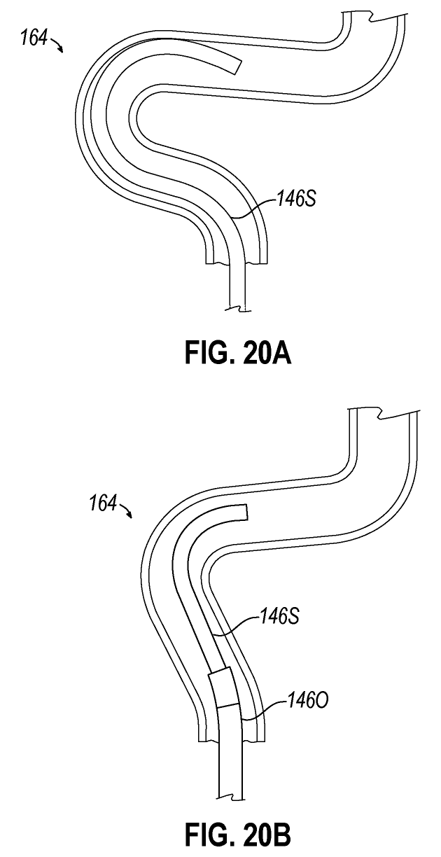

FIGS. 20A-22C depict example operation of the steerable sheath in patient anatomy 164 in accordance with some embodiments.

FIG. 20A depicts an example operation of the scope shaft 146S in a lower GI procedure without a sheath, and FIG. 20B depicts an example operation of the scope shaft 146S in a lower GI procedure with the sheath shaft 146O. As seen in FIG. 20A, pushing the scope 146S without the sheath can create a loop and fail to advance the scope through the pathway (e.g., colon). As seen in FIG. 20B, supporting the scope shaft 146S with the sheath shaft 146O can help advance the scope through the pathway towards a target destination.

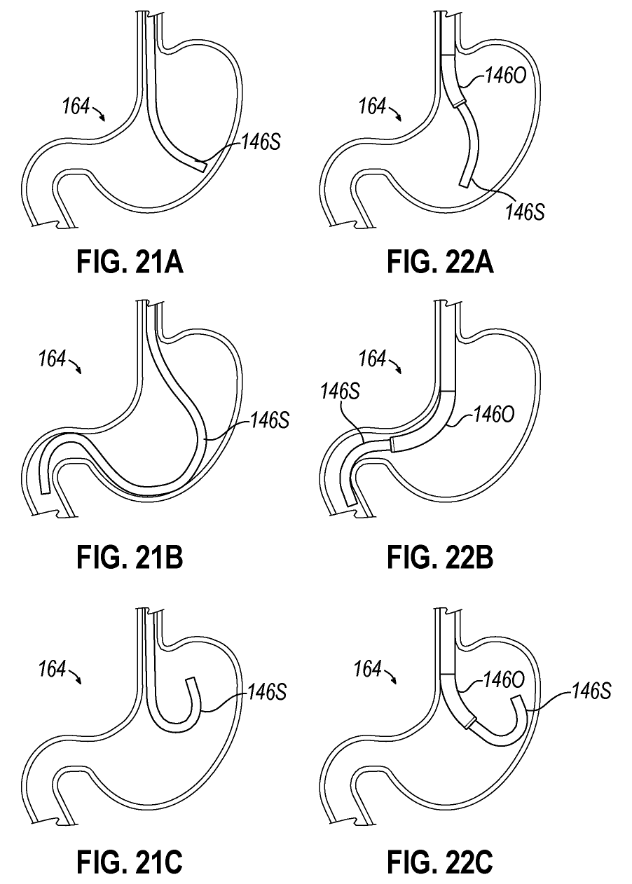

FIGS. 21A-21C depict example operation of the scope shaft 146S in an upper GI procedure without a sheath, and FIGS. 22A-22C depict example operation of the scope shaft 146S in an upper GI procedure with the sheath shaft 146O.

As seen in FIG. 21A and 22A, controlling the scope shaft 146S with the sheath shaft 146O (FIG. 22A) can provide improved support in open spaces compared to controlling the scope without the sheath (FIG. 21A). As seen in FIG. 21B and 22B, controlling the scope shaft 146S with the sheath shaft 146O (FIG. 22B) can provide controlled insertion while mitigating uncontrolled looping or loop formation compared to controlling the scope without the sheath (FIG. 21B). As seen in FIG. 21C and 22C, controlling the scope shaft 146S with the sheath shaft 146O (FIG. 22C) can provide better retroflexion aiming an anatomical access compared to controlling the scope without the sheath (FIG. 21C).

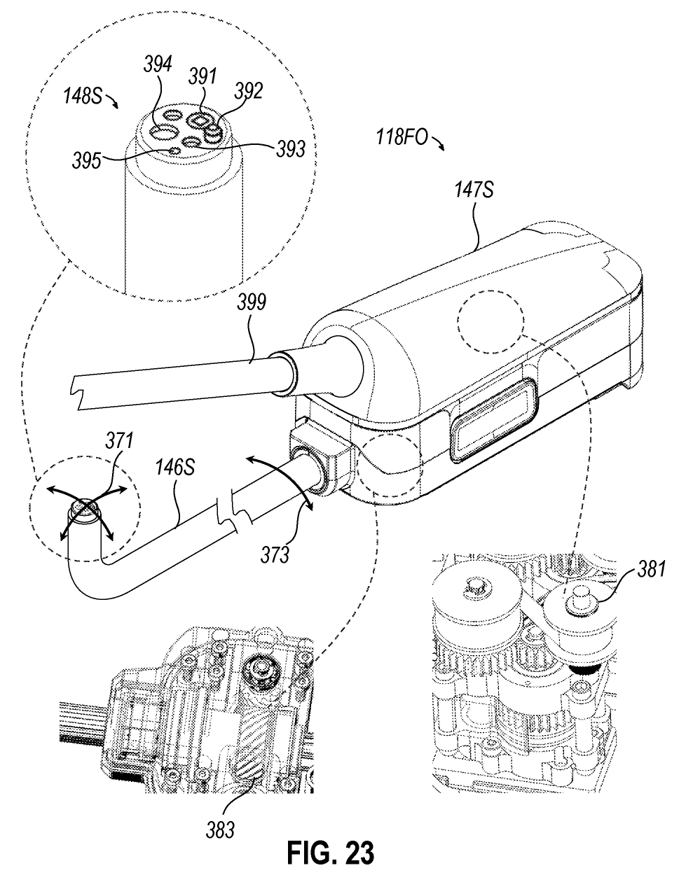

FIG. 23 depicts the scope 118FS as seen in FIG. 18. The scope 118FS has an instrument base 147S and a flexible shaft 146S extending from the instrument base 147S. The instrument base 147S can house mechanisms (e.g., spools or pulleys coupled to pull wires) for steering the tip of the instrument in four steering DOFs 371 (e.g., robotic steering mechanism 381), causing a distal articulating section of the instrument shaft 146S to bend relative to a proximal portion of the instrument shaft 146S and the instrument base 147S. The instrument base 147S can also house mechanisms (e.g., bevel or helical gears) for rolling the scope shaft 146S in roll DOF 373 (e.g., using robotic shaft roll mechanism 383), causing the instrument shaft 146S to rotate about its longitudinal axis (roll axis). Flexible shaft 146S of the scope also includes a working channel or lumen that can receive the shaft of the working channel instrument as noted above. In some variations, the flexible shaft may include localization or tracking sensors, such as shape-sensing fibers along the shaft or electromagnetic sensors in the distal tip. In some variations, the flexible shaft may be provided with variable stiffness along its length.

FIG. 23 also depicts an example configuration of the distal tip 148S of the scope 118FO, in accordance with some embodiments. As seen in FIG. 23, distal tip 148S of scope 118FO can include camera 391 (e.g., stereoscopic or monoscopic) for capturing anatomical images. Camera 391 can include, for instance, an image sensor and optics (e.g., a lens) to facilitate image capture. Distal tip 148S also includes a port of working channel 394 (e.g., a biopsy channel) from which working channel instruments can protrude to obtain biopsy or perform other functions. Distal tip 148S also includes illumination component(s) 393 (e.g., one or more illumination fibers and/or light sources such as LEDs), which can illuminate the scene to facilitate image capture with the camera 391. An insufflation and camera wash component 392 can facilitate insufflation of an anatomical cavity and/or cleaning a lens of the camera 391. Distal tip 148S can also include a water jet 395.

As illustrated, an umbilical cable 399 can be coupled to scope base 147S. Umbilical cable 399 can include electrical cables for power and/or data transmission (e.g., to or from tower 140 of FIG. 1), and in some variations, may also include optical fibers for piping light to the distal tip (e.g., illumination component(s) 393) from a light source or light engine (e.g., from tower 140 of FIG. 1). In the illustrated example, the umbilical cable 399 extends distally from the scope base 147S, in a same or similar direction as the scope shaft 146S. This may help avoid undue bends in the umbilical cable 399 by pointing the cable downwards when the scope base 147 is in an elevated configuration (e.g., loaded state of FIG. 13A).

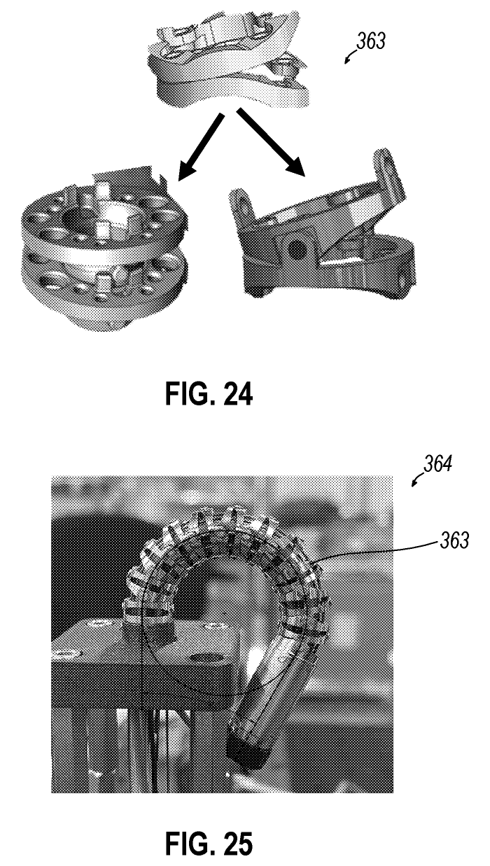

FIG. 24 depicts an example set of vertebrae structures 363 that may be employed in a bending section (also referred to as an “articulating section”) in a distal portion of the scope shaft 146S from FIG. 23. FIG. 25 depicts an example of a bending section 364 of the scope in a bent state using vertebrae structures 363. As illustrated, the bending section 364 can include a series of the vertebrae structures hingedly or pivotably coupled together to facilitate a continuum of bending. In the illustrated configuration, the bending section 364 is bent into a retroflexion state.

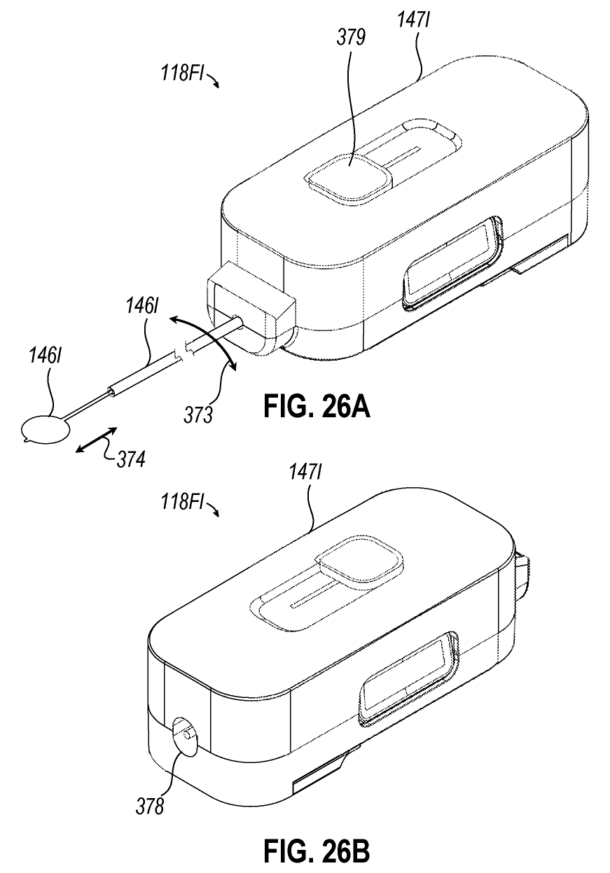

FIGS. 26A-26B depicts the working channel instrument 118FI as seen in FIG. 18, where FIG. 26A is a front perspective view and FIG. 26B is a rear perspective view. The working channel instrument 118FI has an instrument base 147I and a flexible shaft 146I extending from the instrument base 147I. The instrument base 147I can house mechanisms for actuating the instrument, such as actuating the end effector 146I in an actuation DOF 374 and/or rolling the instrument shaft 146I in roll DOF 373, causing the instrument shaft 146I to rotate about its longitudinal axis (roll axis). In addition to robotic drive inputs as described above (e.g., drive inputs 156 of FIG. 9B), working channel instrument can include a manual actuator 379 (also referred to as an off-robot actuator), which in the illustrated example, is configured as a slider. In some variations, as seen in FIG. 26B, the base 147I can include a port 378, such as an electrocautery port (for receiving an electrical cable for energy delivery) or a syringe port (for receiving an injection needle), which can be positioned at a proximal or rear portion of the instrument base 147I.

As described herein, the system can drive flexible working channel instrumentation (tools) that pass through the working channel of an endoluminal scope (colonoscope, gastroscope, etc.). In some embodiments, the system includes a motorized “finger” on the back of a robotic tool drive carriage that moves linearly to push/pull on a slider that is part of the instrument handle. This allows the robot to interface with the instrumentation in a similar manner to how people would use it. In some embodiments, the finger has features to automatically latch on to the instrument’s actuation plunger or slider. This allows for simple flexible working channel instrumentation to be robotically driven while minimizing the added complexity to the instrument handle. Because it interfaces with the handle in a similar manner to manual tools, it also allows the tools to continue to be actuated manually when off-robot.

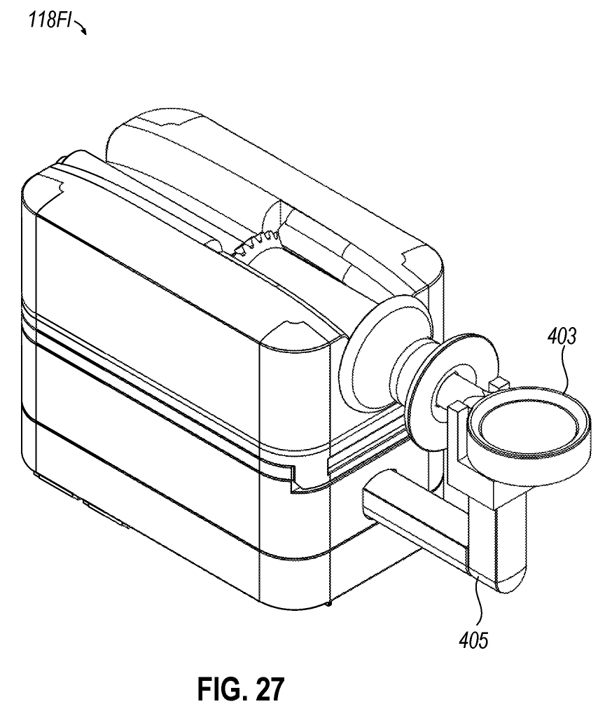

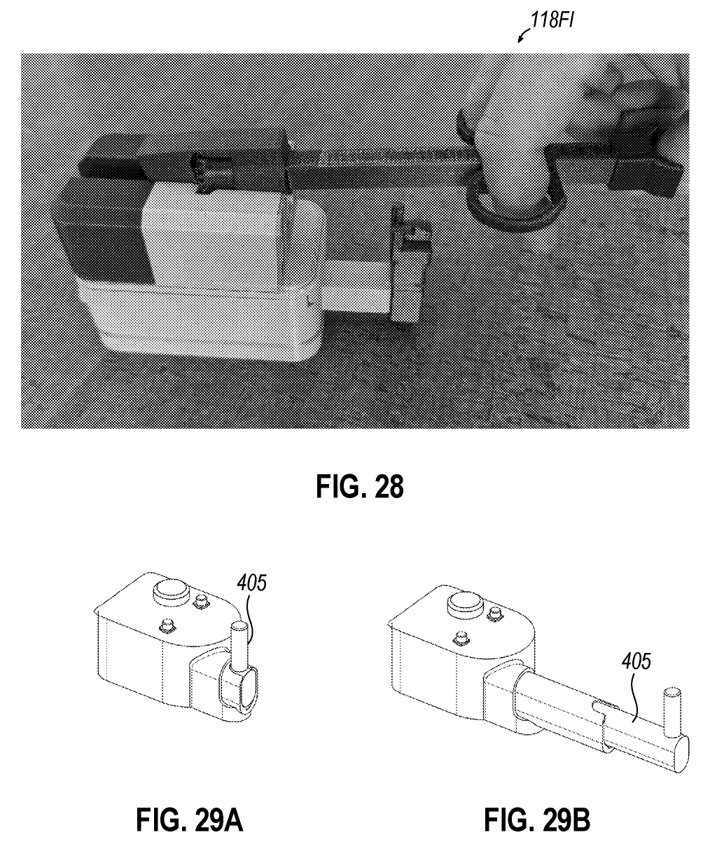

FIGS. 27-29B depict example variations of working channel instrument 118FI employing an actuation finger 405. FIG. 27 depicts working channel instrument 118FI as seen in the set of flexible instruments of FIG. 12. FIG. 28 depicts an example of manual operation of the working channel instrument 118FI. FIGS. 29A-29B depict actuation finger 405 in a contracted state and an expanded state, respectively. As illustrated, the actuation finger 405 can interact with a manual finger loop actuator 403 of working channel instrument, for instance as seen in a standard off the shelf instrument designed for manual operation.

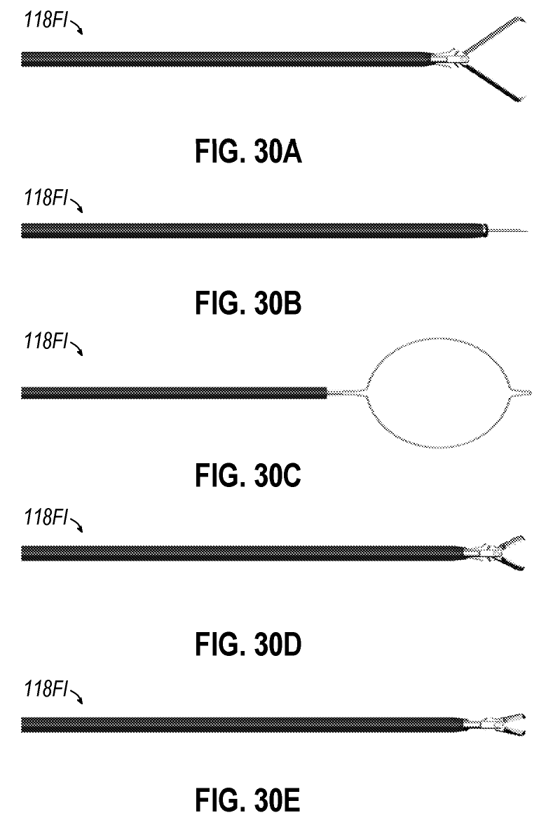

FIGS. 30A-30E depict example variations of working channel instrument 118FI having various types of end effectors, in accordance with some embodiments. FIG. 30A depicts an example of working channel instrument 118FI having a grasper. FIG. 30B depicts an example of working channel instrument 118FI having an injection needle. FIG. 30C depicts an example of working channel instrument 118FI having a snare. FIG. 30D depicts an example of working channel instrument 118FI having biopsy forceps. FIG. 30E depicts an example of a working channel instrument 118FI having hemostatic forceps. In some variations, the instrument base of the working channel instrument can be design to have a common interface with the robot for the various different types of end effectors, allowing the robot to seamlessly actuate different types of working channel instruments as desired for various procedural tasks.

As noted above, a feedroller assembly having one or more feedroller wheels can be employed to advance (insert) or retract (withdraw) a flexible shaft (e.g., translation of the shaft distally or proximally along its longitudinal axis). In some variations, a feedroller assembly is further configured to roll the shaft (e.g., rotate or spin the shaft about a roll axis that coincides with the longitudinal axis). Examples of feedroller assemblies are described below that can drive both translation of the shaft along the shaft axis and rotation of the shaft about the axis, in some instances simultaneously to achieved combined rotation and translation motion. Such feedrollers may be incorporated into a surgical system and driven by a robotic manipulator as described above (e.g., by one or more motors or actuators of the robot), or feedroller assembly may be used in connection with any suitable medical system to drive any instrument shaft, such as scopes, working channel tools, guidewires, or the like.

FIGS. 31A-31C show an example of feedroller assembly 178 engaged with an instrument shaft 146 in operation. As seen in FIG. 31A, feedroller assembly 178 includes a housing 505 and an assembly of multiple wheels 595 retained in the housing 505. In the illustrated example, four wheels 595 (also referred to as “wheel rollers”) are arranged in a radially symmetric arrangement about an axis 599. In some variations, feedroller assembly 178 includes a different number of wheels, such as two, three, or four wheels. Axis 599 can be positioned as a central axis about which the wheels 595 are arranged, and axis 599 can define both an insertion axis and roll axis for the axis 599 that corresponds to the longitudinal axis of the shaft when the shaft is positioned between the wheels 595. The housing 505 includes an opening to permit the shaft to be positioned between the wheels 595, and wheels may 595 frictionally engage or contact the shaft (e.g., clamp or press against the shaft with appropriate force) to drive motion of the shaft.

As seen FIG. 31B, feedroller assembly 178 can translate the shaft 146 along axis 599, for instance, to advance the shaft distally in the direction of the arrow (e.g., to insert the shaft 146 further into a patient’s body). Such motion can be achieved by rotating the wheels about their respective axles. In some variations, all wheels are actively driven in unison to effect translation of the shaft. In some variations, one or more wheels are actively driven, and one or more of the wheels are passive wheels that are free to rotate about their respective axles upon motion of the shaft effected by the actively driven wheel(s). The wheel(s) 595 may be driven in a first direction to drive translation of the shaft in a first direction (e.g., to advance the shaft distally in the direction of arrow seen in FIG. 31B), and the wheel(s) may be driven in a second direction opposite the first direction to drive translation of the shaft in the second direction opposite the first direction (e.g., to retract the shaft proximally in an opposite direction to the arrow seen in FIG. 10B).

As seen FIG. 31C, feedroller assembly 178 can also rotate the shaft 146 about axis 599, for instance, to roll the shaft in the direction of the arrow (e.g., to change an orientation of the tip of the instrument within patient’s body). Such motion can be achieved by collectively rotating the wheel assembly or wheel arrangement about axis 599 so that the wheels collectively rotate together about the central axis (also referred to herein as revolving or orbiting motion). The wheel arrangement may be rotated in a first direction to drive rotation of the shaft in a first direction (e.g., to roll the shaft clockwise in the direction of arrow seen in FIG. 31C), and the wheel arrangement may be rotated in a second direction opposite the first direction to drive rotation of the shaft in the second direction opposite the first direction (e.g., to roll the shaft counterclockwise in an opposite direction to the arrow seen in FIG. 31C). In some variations, a differential gear train or mechanism is utilized by the feedroller assembly 178 to allow the feedroller assembly 178 to effect simultaneous rotation and translation (e.g., rotate the shaft as the shaft is translated without interrupting the translation of the shaft, or translate the shaft as the shaft is rotated without interrupting the rotation of the shaft).

While the examples described herein a feedroller assembly 178 to drive movement of a flexible shaft based on frictional engagement by a set of feedroller wheels, it is contemplated that other modalities may be used to advance, retract, or control movement of the shaft from a distal portion of the flexible shaft. For instance, in any of the examples of systems, manipulators, and endodrivers described herein, the feedroller assembly 178 can be replaced by another type of feed assembly operable to advance, retract, or roll the instrument shaft. Example of feed assemblies included magnetic based feeder mechanisms that use electronically controlled electromagnetic fields to interact with the instrument shaft to drive movement of the shaft, gear-based drive mechanisms that mesh with gear teeth or features on the instrument shaft to drive movement of the shaft, and pinch-and-release type feeder mechanisms that use alternating or reciprocating pinch and release movements to drive movement of the shaft.

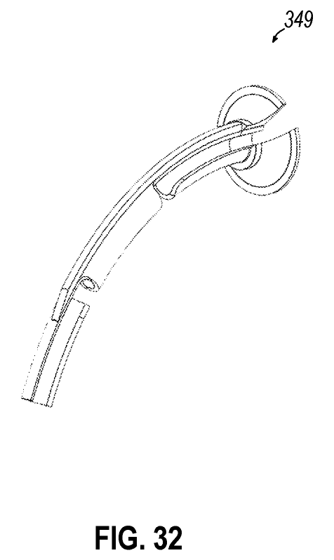

FIG. 32 depicts an example of an introducer 349. As noted above with respect to FIG. 8, the introducer 349 can be coupled to or positioned proximate to the distal assembly 130 to facilitate introducing the instrument shafts into the anatomical opening of the patient. For instance, introducer 349 can facilitate access for an upper GI procedure when the patient is supine.

Introducer 349 can facilitate insertion of a flexible shaft into the patient while, for instance, mitigating buckling of the shaft. Feedroller assembly 178 (e.g., FIGS. 8, 31A-C) can be aligned to the introducer so that an opening of the introducer 349 is aligned with the opening of the feedroller assembly 178, such that the flexible shaft can enter the introducer channel upon exiting the feedroller assembly. As illustrated, introducer 349 can be a rigid curved component that guides a flexible shaft controlled by the feedrollers along a curved path. In some variations, introducer can be straight or curved, and can be rigid or flexible.

FIG. 32 depicts an example of an introducer 349. As noted above with respect to FIG. 8, the introducer 349 can be coupled to or positioned proximate to the distal assembly 130 to facilitate introducing the instrument shafts into the anatomical opening of the patient. For instance, introducer 349 can facilitate access for an upper GI procedure when the patient is supine.

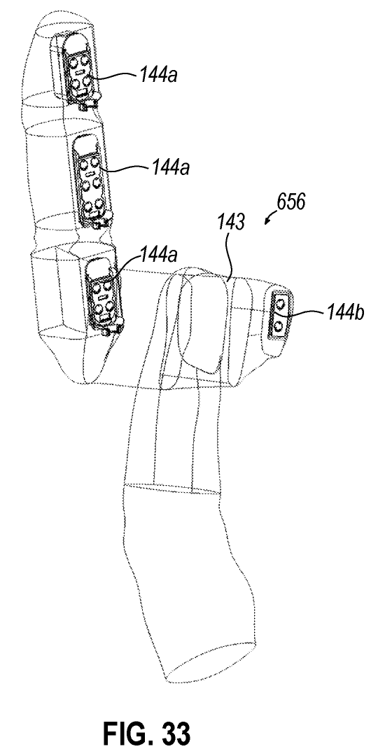

FIG. 33 depicts an example of a barrier 656 configured to cover an endodriver. As illustrated, barrier 656 includes a drape 143 coupled to one or more instrument adapters 144a and one or more feedroller adapters 144b. The drape 143 is configured as a sleeve configured to cover the endodriver arm segment, where the sleeve is coupled to multiple adapters for transferring actuation forces to the respective tools. As illustrated, instrument adapters 144a (e.g., three adapters for the three flexible instruments 118FI, 118FS, 118FO) can be configured to support flexible instruments and transfer actuation forces to the instruments via drive couplers as described above. Feedroller adapter 144b is configured to transfer actuation forces to the feedroller assembly via drive couplers in a similar fashion.

Various principles of this technology are described with reference to laparoscopic procedures, where surgical instruments are introduced to a patient’s abdomen through laparoscopic incisions, and endoluminal procedures, where surgical instruments are introduced through natural orifices. In some variations, a configuration of a surgical system and/or a method of use can involve various types of procedures, anatomical locations, and/or anatomical openings for introducing instruments into a body. Various aspects of the subject matter described herein may be applied to, for instance, purely laparoscopic procedures, purely endoluminal procedures, purely thoracic procedures, open procedures, procedures involving only flexible instruments, procedures involving only rigid instruments, and/or procedures involving any combination of two or more of these approaches.

Various principles of this technology are described with reference to rigid and flexible instrumentation, where, for instance, rigid instruments are introduced through incisions or laparoscopic ports, and flexible instruments are introduced through natural orifices or endoluminal access points. In various configurations, any one or more of these instruments may be flexible or rigid. For instance, in some variations, one or more rigid instruments or rigid scopes may be introduced through a natural orifice. Additionally or alternatively, in some variations, more flexible instruments or flexible scopes may be introduced through an incision or a laparoscopic port.

Various examples disclosed herein describe usage of a surgical system to perform a procedure on a patient, wherein instruments are inserted into a body of the patient. In various configurations, the system may be used, for instance, in educational or lab settings, where a body portion of a model, cadaver, animal, or inanimate object is placed upon the headrest. Such methods may be useful for surgeon training, product testing, development applications, or the like. Accordingly, it will be understood that methods described herein are not limited to medical procedures performed on a human body but can be implemented on bodies or objects that are not part of a live patient or human.

Use of “or” is intended in the inclusive rather than exclusive sense, unless explicitly stated otherwise or the context clearly dictates otherwise. Thus, for example, reference to “A” or “B” can encompass “A” only, “B” only, or both “A” and “B.” As another example, reference to “A, B, or C” can encompass “A” only, “B” only, “C” only, or any combination of two or more of “A” or “B” or “C.” Accordingly, the term “or” should be generally understood as equivalent to “and/or” unless stated otherwise or the context clearly dictates to the contrary.

It should be appreciated that any specific order of steps shown or described herein is illustrative in nature and should not be construed as required unless explicitly stated or the context clearly dictates otherwise. Thus, for example, with respect to any processes or methods herein, any two or more steps or stages in a method or process may performed serially or in parallel, in any combination, and may be performed in any order, unless explicitly stated or the context clearly dictates otherwise.

In some instances, relative positions or orientations are used, such as top, bottom, upper, lower, forward, backward, front, rear, left, right, up down, horizontal, vertical, longitudinal, lateral, or the like. These terms may be used to refer to an arbitrary frame of reference or a frame of reference shown in the drawings, for purposes of explanation or to demonstrate the relative spatial configurations associated with various elements. These terms should not be understood to require any particular gravitational or other frame of reference unless explicitly stated or the context clearly dictates otherwise.

To the extent any headings are used through this description, these headings are used for convenience only and should not be construed as limit the scope of disclosure or the description under a heading to only the topic associated with the heading in anyway.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various examples, configurations, or embodiments of the present technology, further adaptations of the systems or methods described herein may be accomplished by appropriate modifications by one of ordinary skill in the art without departing from the scope of the technology described herein. Several of such potential modifications have been mentioned, and others will be apparent to those skilled in the art. For instance, the examples, embodiments, geometrics, materials, dimensions, ratios, steps, and the like

discussed above are illustrative and are not required. Accordingly, the scope of the claimed subject matter should be considered in terms of the following claims and is understood not to be limited to the details of structure and operation shown and described in the specification and drawings.

What is claimed is:

1. A system comprising:
a flexible instrument comprising an instrument base and a flexible shaft;
a feedroller assembly comprising one or more wheels;
a robot comprising a first link and a second link, the first link configured to support the instrument base, and the second link configured to support the feedroller assembly; and
a control system comprising a processor and memory, the control system configured to:
advance the flexible shaft via operation of the feedroller assembly;
rotate the instrument base relative to the feedroller assembly via rotational movement of the first link relative to the second link; and
advance the instrument base relative to the feedroller assembly via translational movement of the first link relative to the second link.

2. The system of claim 1, the control system configured to move the robot between a plurality of link configurations, the plurality of link configurations including:
a first link configuration where the first link is in a transverse orientation relative to the second link, and the first link is in a retracted position relative to the feedroller assembly; and
a second link configuration where the first link is in a parallel orientation relative to the second link, and the first link is in an advanced position relative to the feedroller assembly.

3. The system of claim 1, the control system configured to move the flexible instrument between a plurality of instrument configurations, the plurality of instrument configurations including:
a first instrument configuration where the instrument base is in an elevated position relative to the feedroller assembly, and the flexible shaft has a service loop portion between the instrument base and the feedroller assembly; and
a second instrument configuration where the instrument base is in a level position relative to the feedroller assembly, and the flexible instrument shaft is taut between the instrument base and the feedroller assembly.

4. The system of claim 1, the first link being coupled to the second link via at least one joint, the at least one joint controlling the rotational movement of the second link relative to the first link about a first rotation axis, the at least one joint controlling the translational movement of the first link relative to the second link along a second linear axis perpendicular to the first rotation axis.

5. The system of claim 1, the robot comprising an instrument carriage mounted to the first link, the instrument carriage configured to support the instrument base, the control system configured to:
advance the instrument base along the first link via translational movement of the instrument carriage along the first link.

6. The system of claim 1, further comprising:
a second flexible instrument comprising a second instrument base and a second flexible shaft;
the robot further comprising a third link configured to support the second instrument base;
the control system configured to:
advance the second instrument base relative to the first instrument base via translational movement of the third link relative to the first link.

7. The system of claim 1, further comprising:
the flexible instrument being one of a set of three flexible instruments arrangeable in a telescoping arrangement;
the control system configured to:
advance respective flexible shafts of the three flexible instruments via the operation of the feedroller assembly;
rotate respective instrument bases of the three flexible instruments relative to the feedroller assembly via the rotational movement of the first link relative to the second link; and
advance the respective instrument bases of the three flexible instruments relative to the feedroller assembly via the translational movement of the first link relative to the second link.

8. The system of claim 1, further comprising:
the robot comprising a deployment arm coupled to the second link and to an operating table, the operating table having a table top, the deployment arm comprising a series of links interconnected by joints;
the control system configured to:
move the first and second links from a stowed position to beneath the table top to a deployed position above the table top via actuation of the deployment arm; and
maintain the deployment arm and the second link stationary during the rotational movement and the translational movement of the first link relative to the second link.

9. The system of claim 1,
the flexible shaft rotatably coupled to the instrument base;
the control system configured to:
roll the flexible shaft about a roll axis relative to the instrument via actuation of a roll mechanism in the flexible instrument and concurrent rotation of the one or more feedrollers about the roll axis.

10. The system of claim 1, further comprising:
a barrier, the barrier comprising:
a drape configured to cover the first link and the second link;
a first adapter mountable to the first link and operable to transfer actuation forces to the flexible instrument; and
a second adapter mountable to the second link and operable to transfer actuation forces to the feedroller assembly.

11. The system of claim 1, further comprising:
an introducer comprising a channel for receiving the flexible shaft.

12. The system of claim 1, further comprising an actuation finger operable to actuate a finger loop of a working channel instrument.

13. The system of claim 1, the rotational movement of the first link relative to second link being about a first axis, the control system configured to:
rotate the instrument base relative to the feedroller assembly about a second axis orthogonal to the first axis via rotational movement of the first link relative to the second link about the second axis.

14. The system of claim 1,
the first link being part of a first kinematic chain;
the second link being part of a second kinematic chain non-serial with respect to the first kinematic chain.

15. A robotic manipulator comprising:
a first link comprising a feedroller coupling portion, the feedroller coupling portion configured to support a feedroller assembly; and
a second link comprising an instrument coupling portion, the instrument portion configured to support an instrument base, the first link being rotatably coupled to the first link such that the first link is rotatable relative to the second link, the first link being translatably coupled to the second link such that the first link is translatable along the second link.

16. The robotic manipulator of claim 15, the first and second links being movable between a plurality of link configurations, the plurality of link configurations including:
a first link configuration where the first link is in a transverse orientation relative to the second link, and the first link is in a retracted position relative to the feedroller assembly; and
a second link configuration where the first link is in a parallel orientation relative to the second link, and the first link is in an advanced position relative to the feedroller assembly.

17. The robotic manipulator of claim 15, the first and second links being movable between a plurality of instrument configurations, the plurality of instrument configurations including:
a first instrument configuration where the instrument coupling portion is in an elevated position relative to the feedroller coupling portion; and
a second instrument configuration where the instrument coupling portion is in a level position relative to the feedroller coupling portion, and the flexible instrument shaft is taut between the instrument base and the feedroller assembly.

18. The robotic manipulator of claim 15, the first link being coupled to the second link via a rotational joint and a linear joint, the rotational joint controlling the rotational movement of the second link relative to the first link about a first rotation axis, the at linear joint controlling the translational movement of the first link relative to the second link along a second linear axis perpendicular to the first rotation axis.

19. The robotic manipulator of claim 15, the first link being configured to actuate a set of three flexible instruments arranged in a telescoping arrangement.

20. A method comprising:
advancing a shaft of a flexible scope via a set of feedrollers, the flexible scope having a handle mounted to a hull;
pivoting the hull about a rotary joint to thereby pivot the handle of the flexible scope relative to the set of feedrollers; and
translating the hull along a linear joint to thereby advance the handle toward the set of feedrollers.
